(19) United States
(12) Patent Application Publication
(10) Pub. No.: US 2026/0102089 A1
(43) Pub. Date: Apr. 16, 2026

(54) Systems and Methods for Skin Assessment with Optical Spectroscopy

(71) Applicant: University of Virginia Patent Foundation, Charlottesville, VA (US)

(72) Inventors: Amanda Watson, Charlottesville, VA (US); Tarek Hamid, Freehold, NJ (US); Kyle Quinn, Charlottesville, VA (US); Anush Niranjan Lingamoorthy, Philadelphia, PA (US)

(21) Appl. No.: 19/356,041

(22) Filed: Oct. 10, 2025

Related U.S. Application Data
(60) Provisional application No. 63/705,967, filed on Oct. 10, 2024.

(51) Int. Cl. A61B 5/1495; A61B 5/00; A61B 5/1455

(52) U.S. Cl. CPC A61B 5/1495; A61B 5/14552; A61B 5/7203; A61B 5/7225; A61B 5/7267

(57) ABSTRACT

A system for collecting skin tone data for a subject uses a plurality of light emitting diodes (LEDs) directing a range of light wavelengths onto skin. A corresponding set of photodiodes (PDs) capture spectral data for a corresponding range of wavelengths from reflected light from the skin. A computer with a processor connects to computer memory storing software that when executed performs a computer implemented method comprising steps of extracting features from the spectral data with a signal processing component of the software and applying the features from the spectral data to a machine learning model that identifies physiology data and color tone data for the skin.

### CROSS-REFERENCE TO RELATED APPLICATIONS

This application claims priority to and the benefit of U.S. provisional Ser. No. 63/705,967, filed on Oct. 10, 2024, and entitled Objective Quantification of Melanin, Erythema and Skin-tone Using Wearable Optical Spectroscopy, the disclosure of which is hereby incorporated by reference herein in its entirety.

### STATEMENT OF GOVERNMENT RIGHTS

None.

### BACKGROUND

Accurate characterization of the skin is essential for optimizing diagnostic and therapeutic dermatological tools, as well as technologies like pulse oximetry that rely on skin perfusion. Traditionally, optical spectroscopy has been used for skin assessments through devices like commercial colorimeters, which are high-cost instruments that, while precise, only provide single measurements rather than continuous data. Additionally, medical wearable devices that use this technology often show variable accuracy based on skin tone. The limitations of existing devices demonstrate the need for a solution that can provide low-cost, accurate, and continuous skin monitoring across varying skin tones in a wearable form-factor.

This disclosure utilizes raw data that is amenable to numerous kinds of mathematical and computer implemented methods of analysis. In some embodiments, artificial intelligence and machine learning techniques may be used in optional embodiments of this disclosure. Machine Learning (ML) and Artificial Intelligence (AI) systems are in widespread use in customer service, marketing, and other industries, including medicine and science. Machine learning is considered a subset of more general artificial intelligence operations, and AI endeavors may utilize numerous instances of machine learning to make decisions, predict outputs, and perform human-like intelligent operations. Machine learning protocols typically involve programming a model that instantiates an appropriate algorithm for a given computing environment and training the model on a particular data set or domain with known historical results. The results are generally known outputs of many combinations of parameter values that the algorithm accesses during training. The model uses numerous statistical and mathematical operations to learn how to make logical decisions and generate new outputs based on the historical training data. Machine learning (ML) includes, but is not limited to, a number of models such as neural networks, deep learning algorithms, support vector machines, data clustering, regression models, and Monte Carlo simulations. Other models may utilize linear regression, logistic regression, support vector machines, K-means clustering, classification models such as a binary classifier or a multi-class classifier, clustering models, anomaly detection, other supervised learning models, and even combinations of one or more machine language model types. Most of these take vectors of data as inputs.

The term “artificial intelligence,” therefore, includes any technique that enables one or more computing devices or comping systems (i.e., a machine) to mimic human intelligence. Artificial intelligence (AI) includes, but is not limited to, knowledge bases, machine learning, representation learning, and deep learning. The term “machine learning” is generally a subset of AI that enables a machine to acquire knowledge by extracting patterns from raw data.

The term “representation learning” may be used as a subset of machine learning that enables a machine to automatically discover representations needed for feature detection, prediction, or classification from raw data. Representation learning techniques include, but are not limited to, autoencoders.

The term “deep learning” may also be considered a subset of machine learning that enables a machine to automatically discover representations needed for feature detection, prediction, classification, etc. using layers of processing. Deep learning techniques include, but are not limited to, artificial neural network or multilayer perceptron (MLP).

Machine learning models include supervised, semi-supervised, and unsupervised learning models. In a supervised learning model, the model learns a function that maps an input (also known as feature or features) to an output (also known as target or target) during training with a labeled data set (or dataset). In an unsupervised learning model, the model learns a function that maps an input (also known as feature or features) to an output (also known as target or target) during training with an unlabeled data set. In a semi-supervised model, the model learns a function that maps an input (also known as feature or features) to an output (also known as target or target) during training with both labeled and unlabeled data.

Some machine learning models are designed for a specific data set or domain and are highly expert at handling the nuances within that narrow domain. It is with respect to these and other considerations that the various aspects of the present disclosure as described below are presented. This disclosure combines algorithms deciphered by artificial intelligence and machine learning with currently known systems and models that gather data from a patient on a real time basis. Accordingly, this disclosure can utilize sensors and medical equipment that improve a system's ability to diagnose and treat a patient.

Brackets with numerals therein refer to references cited the below disclosure.

### SUMMARY

In one embodiment, a system for collecting skin tone data for a subject includes a plurality of light emitting diodes (LEDs) directing a range of light wavelengths onto skin. A corresponding set of photodiodes (PDs) capture spectral data for a corresponding range of wavelengths from reflected light from the skin. A computer having a processor connected to computer memory stores software that when executed performs a computer implemented method with steps of extracting features from the spectral data with a signal processing component of the software and applying the features from the spectral data to a machine learning model that identifies physiology data and color tone data for the skin.

In another embodiment, the photodiodes capture the spectral data on a continuous basis.

In another embodiment, extracting features includes calculating mean values of each photodiode response over a measurement period.

In another embodiment, extracting features includes calculating direct current (DC) content of each PD response over a measurement period.

In another embodiment, extracting features includes calculating an individual typology angle (ITA) for the skin.

In another embodiment, extracting features includes calculating ratios of combinations of LED outputs and photodiode responses.

In another embodiment, combinations of LED outputs and photodiode responses different wavelengths of light.

In another embodiment, the range of wavelengths of the LEDs is within the visible spectrum.

In another embodiment, the spectral data is three-dimensional spectral data.

In another embodiment, the three-dimensional spectral data includes an L* axis, and a* axis, and a b* axis.

In another embodiment, extracting features includes calculating an individual typology angle (ITA) for the skin with L* and b* components from the three-dimensional spectral data.

In another embodiment, the individual typology angle (ITA) is used to classify the color tone of the skin according to an ITA range.

In another embodiment, the machine learning model identifies the physiology data of the skin with a regression algorithm.

In another embodiment, the regression algorithm assigns a quantitative value to either or both of melanin level or erythema level of the skin.

In another embodiment, the system further comprising a pulse oximeter that is calibrated according to at least one of a skin color tone, a melanin level, or an erythema level.

### BRIEF DESCRIPTION OF THE DRAWINGS

Reference will now be made to the accompanying drawings, which are not necessarily drawn to scale.

FIG. 1A is a high-level flow chart of method of conducting skin analysis for use with additional hardware as described herein.

FIG. 1B is a flow chart of one embodiment of a computer implemented method that may be used according to this disclosure to conduct skin analysis for use with medical equipment as disclosed herein.,

FIG. 2 is an overview of a computing environment capable of utilizing machine learning processes to process skin data according to this disclosure

FIG. 3A is a computer architecture diagram showing a computing system capable of implementing aspects of the present disclosure in accordance with one or more embodiments.

FIG. 3B is a computer architecture diagram showing a networking environment that allows for data communication with a computing system capable of implementing aspects of the present disclosure in accordance with one or more embodiments.

FIG. 4 is a block diagram that illustrates a system 130 including a computer system 140 and the associated Internet 11 connection upon which an embodiment may be implemented.

FIG. 5 illustrates a system in which one or more embodiments of the disclosure can be implemented using a network, or portions of a network or computers. The present disclosure may be practiced with or without a network.

FIG. 6 illustrates an embodiment that includes, but is not limited thereto, a system, method, and computer readable medium that provides for utilizing machine learning processes to process digital images and map a selected anatomy of a subject.

FIG. 7 illustrates an advanced framework that utilizes wearable, multi-wavelength optical spectroscopy to deliver low-cost, continuous, and non-invasive measurements of melanin, erythema, and skin-color accurately across various skin tones and body locations.

FIG. 8 is a schematic overview of a framework that utilizes off-the-shelf multi-wavelength optical sensing to predict melanin, erythema, and skin-tone values.

FIG. 9A is a graphical illustration depicting a spectral response of light emitting diodes used in this disclosure.

FIG. 9B is a graphical illustration depicting a spectral response of photodiodes used in this disclosure.

FIG. 10 is a schematic of a process of a data processing, feature engineering, and model training pipeline for pulse oximeter bias correction using OpenOximetry dataset from PhysioNet.

FIG. 11A is a graphical representation of an observation frequency of individual typology angle (ITA) classifications across measurement locations.

FIG. 11B is a graphical representation of observation frequency of individual typology angle (ITA) classifications per subject using a single shoulder measurement.

FIG. 12A is a graphical representation of normalized mean absolute error (NMAE) and ΔE values across target variables grouped by ITA skin-tone classification.

FIG. 12B is a graphical representation of normalized mean absolute error (NMAE) and ΔE values across target variables grouped by ITA skin-tone classification.

FIG. 12C is a graphical representation of normalized mean absolute error (NMAE) and ΔE values across target variables grouped by ITA skin-tone classification.

FIG. 13A is a graphical representation of normalized mean absolute error (NMAE) and ΔE values across target variables grouped by location of measurement.

FIG. 13B is a graphical representation of normalized mean absolute error (NMAE) and ΔE values across target variables grouped by location of measurement.

FIG. 13C is a graphical representation of normalized mean absolute error (NMAE) and ΔE values across target variables grouped by location of measurement.

FIG. 14A is a graphical representation of Mean Absolute Error (MAE) that is decreased in normal conditions for devices with high SpO2 error (>3%) by incorporating skin-tone measurements.

FIG. 14B is a graphical representation of Mean Absolute Error (MAE) that is decreased in hypoxic conditions for devices with high SpO2 error (>3%) by incorporating skin-tone measurements.

FIG. 15A is a graphical representation of mean bias correction factor for pulse oximeters is significantly different between ITA skin-tone classifications. The Mean bias correction factor for all blood oxygen measurements per skin tone group demonstrates significant differences across groups.

FIG. 15B is a graphical representation of the individual typology angle (ITA) distribution for unique patients in the OpenOximetry database using the fingertip measurement. ITA scale distribution for fingertip measurements in the OpenOximetry database, showing a skewed distribution with fewer dark-skinned individuals.

FIG. 16 is a depiction of Table 1 referenced in this disclosure.

FIG. 17 is a depiction of Table 2 referenced in this disclosure.

FIG. 18 is a depiction of Table 3 referenced in this disclosure.

FIG. 19 is a depiction of Table 4 referenced in this disclosure.

FIG. 20 is a depiction of Table 5 referenced in this disclosure.

FIG. 21 is a depiction of Table 6 referenced in this disclosure.

FIG. 22 is a depiction of Table 7 referenced in this disclosure.

FIG. 23 is a depiction of Table 8 referenced in this disclosure.

FIG. 24 is a depiction of Table 9 referenced in this disclosure.

FIG. 25 is a depiction of Table 10 referenced in this disclosure.

### DETAILED DESCRIPTION

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the disclosed technology. Similarly, it is also to be understood that the mention of one or more components in a device or system does not preclude the presence of additional components or intervening components between those components expressly identified.

As discussed herein, a “subject” (or “patient”) may be any applicable human, animal, or other organism, living or dead, or other biological or molecular structure or chemical environment, and may relate to particular components of the subject, for instance specific organs, tissues, or fluids of a subject, may be in a particular location of the subject, referred to herein as an “area of interest” or a “region of interest.”

A detailed description of aspects of the disclosed technology, in accordance with various example embodiments, will now be provided with reference to the accompanying drawings. The drawings form a part hereof and show, by way of illustration, specific embodiments and examples. In referring to the drawings, like numerals represent like elements throughout the several figures. An aspect of an embodiment of the present disclosure provides, among other things, a system, method and computer readable medium for providing a computer implemented paradigm that leverages computing power and associated hardware to perform skin tone analysis. Systems, methods, and products of this disclosure are designed to train a context-aware, personalized algorithm. Enhanced decision-making implemented herein includes analyzing patterns that may not be apparent to humans, but the computer implemented methods of this disclosure can potentially optimize skin analysis for use in associated equipment.

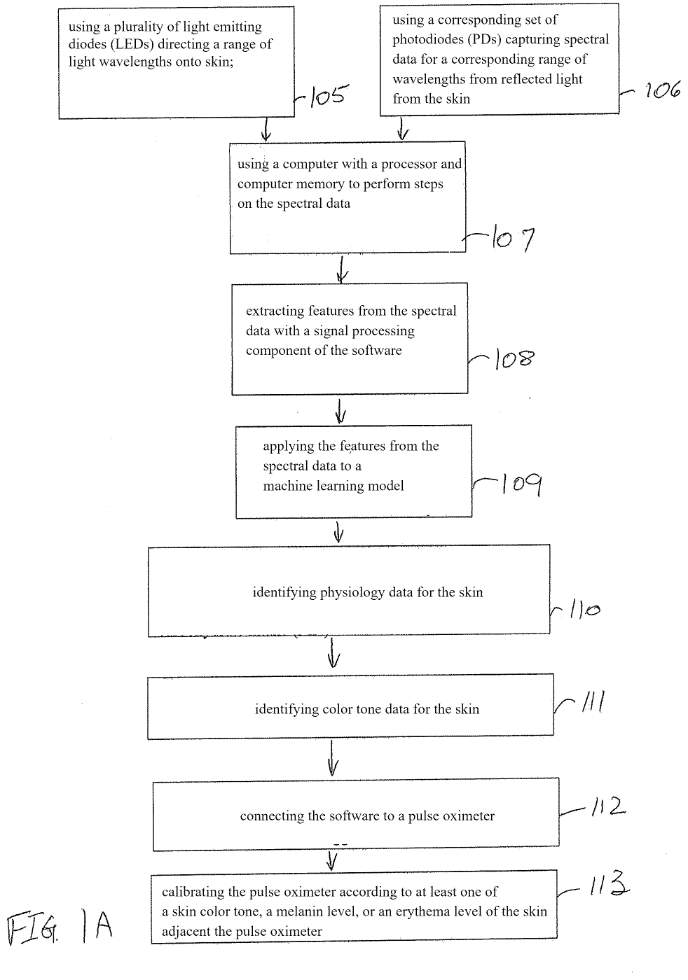

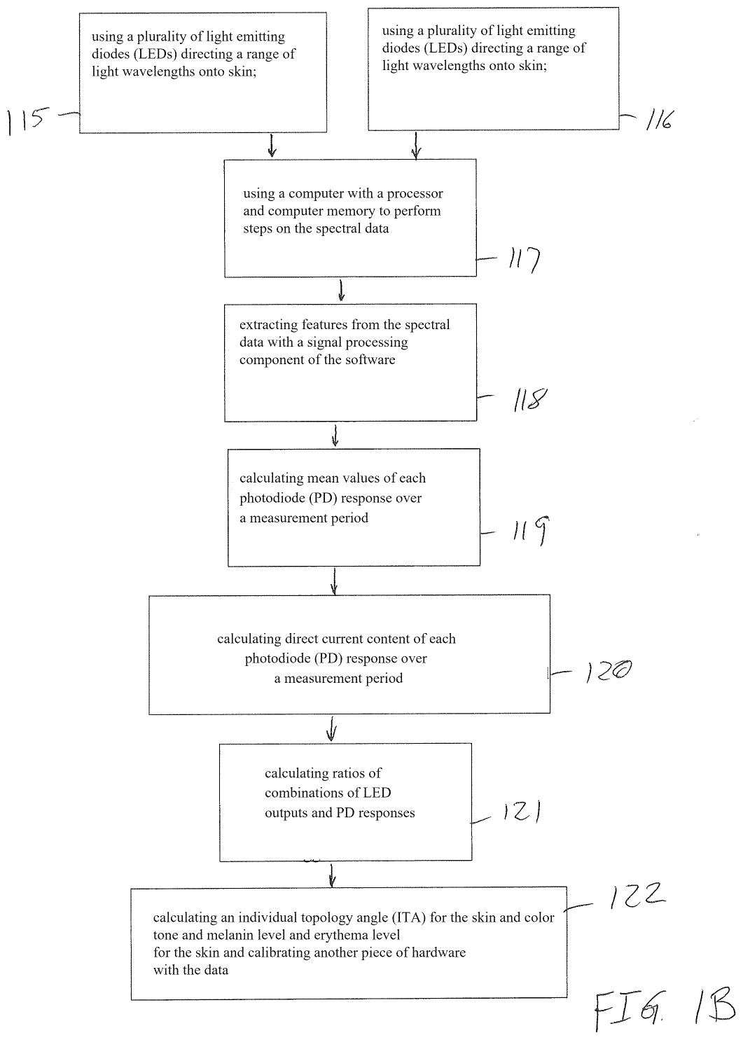

FIG. 1A is a high-level flow chart of method of conducting skin analysis for use with additional hardware as described herein.

FIG. 1B is a flow chart of one embodiment of a computer implemented method that may be used according to this disclosure to conduct skin analysis for use with medical equipment as disclosed herein.,

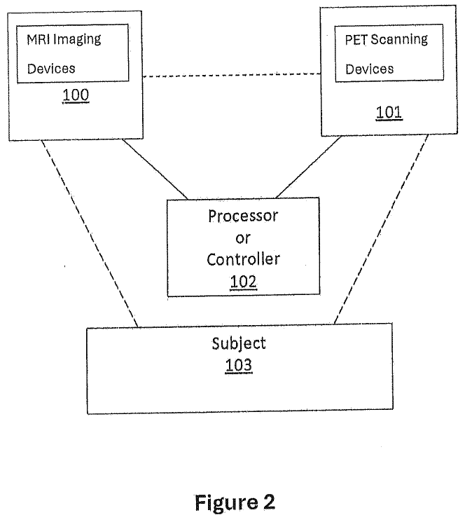

FIG. 2 is a high-level functional block diagram of an embodiment of the present disclosure, or an aspect of an embodiment of the present disclosure. As shown in FIG. 2, a processor or controller 102 communicates with the glucose monitor or device 101, and optionally the insulin device 100. The glucose monitor or device 101 communicates with the subject 103 to monitor glucose levels of the subject 103. The processor or controller 102 is configured to perform the required calculations. Optionally, the insulin device 100 communicates with the subject 103 to deliver insulin to the subject 103. The processor or controller 102 is configured to perform the required calculations. The glucose monitor 101 and the insulin device 100 may be implemented as a separate device or as a single device. The processor 102 can be implemented locally in the glucose monitor 101, the insulin device 100, or a standalone device (or in any combination of two or more of the glucose monitor, insulin device, or a stand along device). The processor 102 or a portion of the system can be located remotely such that the device is operated as a telemedicine device. FIG. 2 also illustrates sensors and detectors that can be used to gather field data measurements for a subject, in real time or from samples, from the patient's blood. These kinds of sensors and detectors may be stand-alone equipment or incorporated into an insulin delivery device or pump.

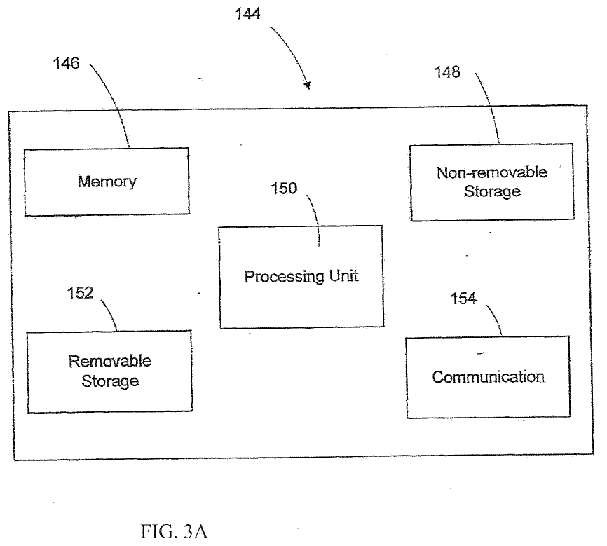

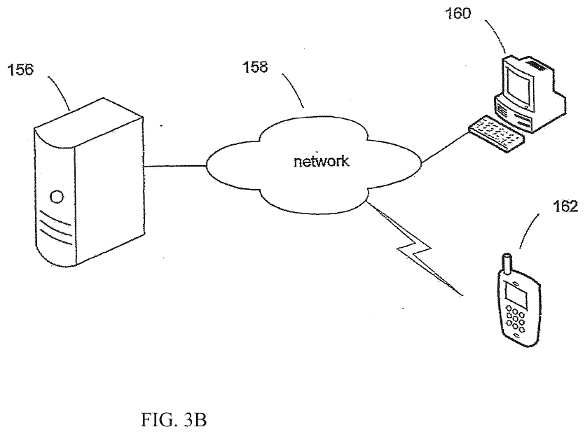

Referring to FIG. 3A, in its most basic configuration, computing device 144 typically includes at least one processing unit 150 and memory 146. Depending on the exact configuration and type of computing device, memory 146 can be volatile (such as RAM), non-volatile (such as ROM, flash memory, etc.) or some combination of the two.

Additionally, device 144 may also have other features and/or functionality. For example, the device could also include additional removable and/or non-removable storage including, but not limited to, magnetic or optical disks or tape, as well as writable electrical storage media. Such additional storage is the figure by removable storage 152 and non-removable storage 148.

Computer storage media includes volatile and nonvolatile, removable and non-removable media implemented in any method or technology for storage of information such as computer readable instructions, data structures, program modules or other data. The memory, the removable storage and the non-removable storage are all examples of computer storage media. Computer storage media includes, but is not limited to, RAM, ROM, EEPROM, flash memory or other memory technology CDROM, digital versatile disks (DVD) or other optical storage, magnetic cassettes, magnetic tape, magnetic disk storage or other magnetic storage devices, or any other medium which can be used to store the desired information and which can accessed by the device. Any such computer storage media may be part of, or used in conjunction with, the device.

The device may also contain one or more communications connections 154 that allow the device to communicate with other devices (e.g. other computing devices). The communications connections carry information in a communication media. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode, execute, or process information in the signal. By way of example, and not limitation, communication medium includes wired media such as a wired network or direct-wired connection, and wireless media such as radio, RF, infrared and other wireless media. As discussed above, the term computer readable media as used herein includes both storage media and communication media.

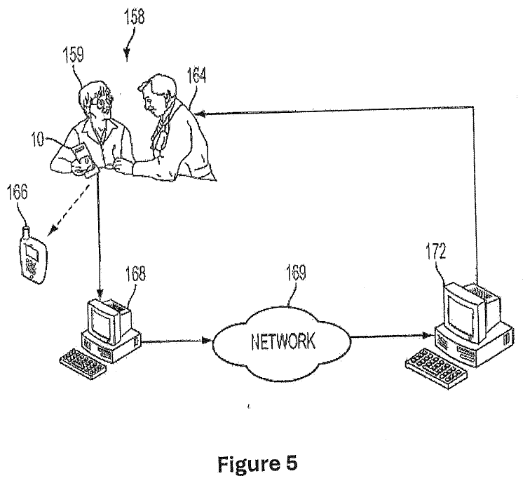

In addition to a stand-alone computing machine, embodiments of the disclosure can also be implemented on a network system comprising a plurality of computing devices that are in communication with a networking means, such as a network with an infrastructure or an ad hoc network. The network connection can be wired connections or wireless connections. As a way of example, FIG. 5 illustrates a network system in which embodiments of the disclosure can be implemented. In this example, the network system comprises computer 156 (e.g. a network server), network connection means 158 (e.g. wired and/or wireless connections), computer terminal 160, and PDA (e.g. a smart-phone) 162 (or other handheld or portable device, such as a cell phone, laptop computer, tablet computer, GPS receiver, mp3 player, handheld video player, pocket projector, etc. or handheld devices (or non-portable devices) with combinations of such features).

In an embodiment, it should be appreciated that the module listed as 156 may be glucose monitor device. In an embodiment, it should be appreciated that the module listed as 156 may be a glucose monitor device, artificial pancreas, and/or an insulin device (or other interventional or diagnostic device). Any of the components shown or discussed with FIG. 3B may be multiple in number.

The embodiments of the disclosure can be implemented in anyone of the devices of the system. For example, execution of the instructions or other desired processing can be performed on the same computing device that is anyone of 156, 160, and 162. Alternatively, an embodiment of the disclosure can be performed on different computing devices of the network system. For example, certain desired or required processing or execution can be performed on one of the computing devices of the network (e.g. server 156 and/or glucose monitor device), whereas other processing and execution of the instruction can be performed at another computing device (e.g. terminal 160) of the network system, or vice versa. In fact, certain processing or execution can be performed at one computing device (e.g. server 156 and/or insulin device, artificial pancreas, or glucose monitor device (or other interventional or diagnostic device)); and the other processing or execution of the instructions can be performed at different computing devices that may or may not be networked.

For example, the certain processing can be performed at terminal 160, while the other processing or instructions are passed to device 162 where the instructions are executed. This scenario may be of particular value especially when the PDA 162 device, for example, accesses the network through computer terminal 160 (or an access point in an ad hoc network). For another example, software to be protected can be executed, encoded or processed with one or more embodiments of the disclosure. The processed, encoded or executed software can then be distributed to customers. The distribution can be in the form of storage media (e.g. disk) or electronic copy.

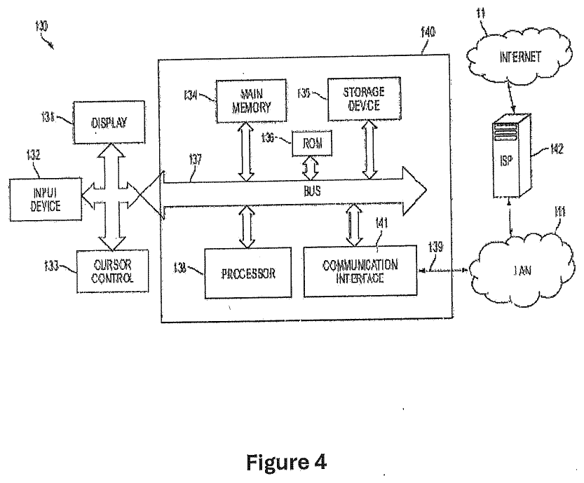

FIG. 4 is a block diagram that illustrates a system 130 including a computer system 140 and the associated Internet 11 connection upon which an embodiment may be implemented. Such configuration is typically used for computers (hosts) connected to the Internet 11 and executing a server or a client (or a combination) software. A source computer such as laptop, an ultimate destination computer and relay servers, for example, as well as any computer or processor described herein, may use the computer system configuration and the Internet connection shown in FIG. 4. The system 140 may be used as a portable electronic device such as a notebook/laptop computer, a media player (e.g., MP3 based or video player), a cellular phone, a Personal Digital Assistant (PDA), a glucose monitor device, an artificial pancreas, an insulin delivery device (or other interventional or diagnostic device), an image processing device (e.g., a digital camera or video recorder), and/or any other handheld computing devices, or a combination of any of these devices. Note that while FIG. 4 illustrates various components of a computer system, it is not intended to represent any particular architecture or manner of interconnecting the components; as such details are not germane to the present disclosure. It will also be appreciated that network computers, handheld computers, cell phones and other data processing systems which have fewer components or perhaps more components may also be used. The computer system of FIG. 4 may, for example, be an Apple Macintosh computer or Power Book, or an IBM compatible PC. Computer system 140 includes a bus 137, an interconnect, or other communication mechanism for communicating information, and a processor 138, commonly in the form of an integrated circuit, coupled with bus 137 for processing information and for executing the computer executable instructions. Computer system 140 also includes a main memory 134, such as a Random Access Memory (RAM) or other dynamic storage device, coupled to bus 137 for storing information and instructions to be executed by processor 138.

Main memory 134 also may be used for storing temporary variables or other intermediate information during execution of instructions to be executed by processor 138. Computer system 140 further includes a Read Only Memory (ROM) 136 (or other non-volatile memory) or other static storage device coupled to bus 137 for storing static information and instructions for processor 138. A storage device 135, such as a magnetic disk or optical disk, a hard disk drive for reading from and writing to a hard disk, a magnetic disk drive for reading from and writing to a magnetic disk, and/or an optical disk drive (such as DVD) for reading from and writing to a removable optical disk, is coupled to bus 137 for storing information and instructions. The hard disk drive, magnetic disk drive, and optical disk drive may be connected to the system bus by a hard disk drive interface, a magnetic disk drive interface, and an optical disk drive interface, respectively. The drives and their associated computer-readable media provide non-volatile storage of computer readable instructions, data structures, program modules and other data for the general purpose computing devices. Typically computer system 140 includes an Operating System (OS) stored in a non-volatile storage for managing the computer resources and provides the applications and programs with an access to the computer resources and interfaces. An operating system commonly processes system data and user input, and responds by allocating and managing tasks and internal system resources, such as controlling and allocating memory, prioritizing system requests, controlling input and output devices, facilitating networking and managing files. Non-limiting examples of operating systems are Microsoft Windows, Mac OS X, and Linux.

The term “processor” is meant to include any integrated circuit or other electronic device (or collection of devices) capable of performing an operation on at least one instruction including, without limitation, Reduced Instruction Set Core (RISC) processors, CISC microprocessors, Microcontroller Units (MCUs), CISC-based Central Processing Units (CPUs), and Digital Signal Processors (DSPs). The hardware of such devices may be integrated onto a single substrate (e.g., silicon “die”), or distributed among two or more substrates. Furthermore, various functional aspects of the processor may be implemented solely as software or firmware associated with the processor. Computer system 140 may be coupled via bus 137 to a display 131, such as a Cathode Ray Tube (CRT), a Liquid Crystal Display (LCD), a flat screen monitor, a touch screen monitor or similar means for displaying text and graphical data to a user. The display may be connected via a video adapter for supporting the display. The display allows a user to view, enter, and/or edit information that is relevant to the operation of the system. An input device 132, including alphanumeric and other keys, is coupled to bus 137 for communicating information and command selections to processor 138. Another type of user input device is cursor control 133, such as a mouse, a trackball, or cursor direction keys for communicating direction information and command selections to processor 138 and for controlling cursor movement on display 131. This input device typically has two degrees of freedom in two axes, a first axis (e.g., x) and a second axis (e.g., y), that allows the device to specify positions in a plane.

The computer system 140 may be used for implementing the methods and techniques described herein. According to one embodiment, those methods and techniques are performed by computer system 140 in response to processor 138 executing one or more sequences of one or more instructions contained in main memory 134. Such instructions may be read into main memory 134 from another computer-readable medium, such as storage device 135. Execution of the sequences of instructions contained in main memory 134 causes processor 138 to perform the process steps described herein. In alternative embodiments, hard-wired circuitry may be used in place of or in combination with software instructions to implement the arrangement. Thus, embodiments of the disclosure are not limited to any specific combination of hardware circuitry and software.

The term “computer-readable medium” (or “machine-readable medium”) as used herein is an extensible term that refers to any medium or any memory, that participates in providing instructions to a processor, (such as processor 138) for execution, or any mechanism for storing or transmitting information in a form readable by a machine (e.g., a computer). Such a medium may store computer-executable instructions to be executed by a processing element and/or control logic, and data which is manipulated by a processing element and/or control logic, and may take many forms, including but not limited to, non-volatile medium, volatile medium, and transmission medium. Transmission media includes coaxial cables, copper wire and fiber optics, including the wires that comprise bus 137. Transmission media can also take the form of acoustic or light waves, such as those generated during radio-wave and infrared data communications, or other form of propagated signals (e.g., carrier waves, infrared signals, digital signals, etc.). Common forms of computer-readable media include, for example, a floppy disk, a flexible disk, hard disk, magnetic tape, or any other magnetic medium, a CD-ROM, any other optical medium, punch-cards, paper-tape, any other physical medium with patterns of holes, a RAM, a PROM, and EPROM, a FLASH-EPROM, any other memory chip or cartridge, a carrier wave as described hereinafter, or any other medium from which a computer can read.

Various forms of computer-readable media may be involved in carrying one or more sequences of one or more instructions to processor 138 for execution. For example, the instructions may initially be carried on a magnetic disk of a remote computer. The remote computer can load the instructions into its dynamic memory and send the instructions over a telephone line using a modem. A modem local to computer system 140 can receive the data on the telephone line and use an infra-red transmitter to convert the data to an infra-red signal. An infra-red detector can receive the data carried in the infra-red signal and appropriate circuitry can place the data on bus 137. Bus 137 carries the data to main memory 134, from which processor 138 retrieves and executes the instructions. The instructions received by main memory 134 may optionally be stored on storage device 135 either before or after execution by processor 138.

Computer system 140 also includes a communication interface 141 coupled to bus 137.

Communication interface 141 provides a two-way data communication coupling to a network link 139 that is connected to a local network 111. For example, communication interface 141 may be an Integrated Services Digital Network (ISDN) card or a modem to provide a data communication connection to a corresponding type of telephone line. As another non-limiting example, communication interface 141 may be a local area network (LAN) card to provide a data communication connection to a compatible LAN. For example, Ethernet based connection based on IEEE802.3 standard may be used such as 10/100BaseT, 1000BaseT (gigabit Ethernet), 10gigabit Ethernet (10 GE or 10 GbE or 10 GigE per IEEE Std 802.3ae-2002 as standard), 40 Gigabit Ethernet (40 GbE), or 100 Gigabit Ethernet (100 GbE as per Ethernet standard IEEE P802.3ba), as described in Cisco Systems, Inc. Publication number 1-587005-001-3 (6/99), “Internetworking Technologies Handbook”, Chapter 7:“Ethernet Technologies”, pages 7-1 to 7-38, which is incorporated in its entirety for all purposes as if fully set forth herein. In such a case, the communication interface 141 typically include a LAN transceiver or a modem, such as Standard Microsystems Corporation (SMSC) LAN91C111 10/100 Ethernet transceiver described in the Standard Microsystems Corporation (SMSC) data-sheet “LAN91C111 10/100 Non-PCI Ethernet Single Chip MAC+PHY” Data-Sheet, Rev. 15 (02-20-04), which is incorporated in its entirety for all purposes as if fully set forth herein.

Wireless links may also be implemented. FIG. 5 illustrates setups 158 in which multiple parties 159, 164 share information across a network 169 with numerous devices that can be a handheld telephone or mobile device 10, 166 or standard computers 168, 172. In any such implementation, communication interface 141 sends and receives electrical, electromagnetic or optical signals that carry digital data streams representing various types of information. Network link 139 typically provides data communication through one or more networks to other data devices. For example, network link 139 may provide a connection through local network 111 to a host computer or to data equipment operated by an Internet Service Provider (ISP) 142. ISP 142 in turn provides data communication services through the world-wide packet data communication network Internet 11. Local network 111 and Internet 11 both use electrical, electromagnetic or optical signals that carry digital data streams. The signals through the various networks and the signals on the network link 139 and through the communication interface 141, which carry the digital data to and from computer system 140, are exemplary forms of carrier waves transporting the information. A received code may be executed by processor 138 as it is received, and/or stored in storage device 135, or other non-volatile storage for later execution. In this manner, computer system 140 may obtain application code in the form of a carrier wave.

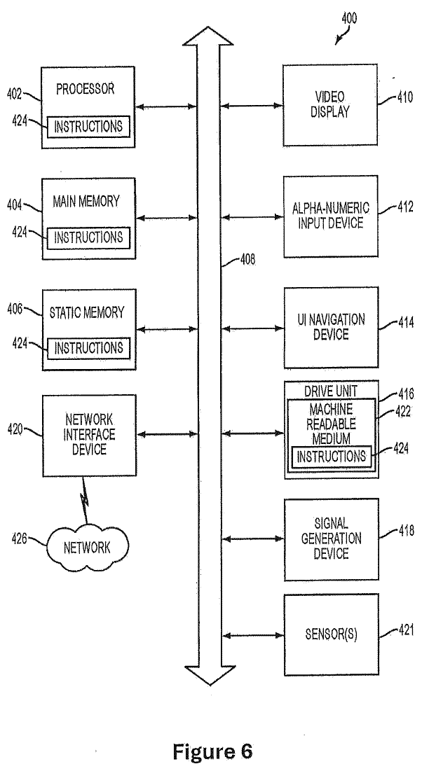

FIG. 6 is a block diagram illustrating an example of a machine upon which one or more aspects of embodiments of the present disclosure can be implemented.

Examples of machine 400 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In embodiments in which multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor-implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a “cloud computing” environment or as a “software as a service” (SaaS). For example, at least some of the operations can be performed by a group of computers (as examples of machines including processors), with these operations being accessible via a network (e.g., the Internet) and via one or more appropriate interfaces (e.g., Application Program Interfaces (APIs).)

Example embodiments (e.g., apparatus, systems, or methods) can be implemented in digital electronic circuitry, in computer hardware, in firmware, in software, or in any combination thereof. Example embodiments can be implemented using a computer program product (e.g., a computer program, tangibly embodied in an information carrier or in a machine-readable medium, for execution by, or to control the operation of, data processing apparatus such as a programmable processor, a computer, or multiple computers).

A computer program can be written in any form of programming language, including compiled or interpreted languages, and it can be deployed in any form, including as a stand-alone program or as a software module, subroutine, or other unit suitable for use in a computing environment. A computer program can be deployed to be executed on one computer or on multiple computers at one site or distributed across multiple sites and interconnected by a communication network. In an example, operations can be performed by one or more programmable processors executing a computer program to perform functions by operating on input data and generating output. Examples of method operations can also be performed by, and example apparatus can be implemented as special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine 400) and software architectures that can be deployed in example embodiments.

In an example, the machine 400 can operate as a standalone device or the machine 400 can be connected (e.g., networked) to other machines.

In a networked deployment, the machine 400 can operate in the capacity of either a server or a client machine in server-client network environments. In an example, machine 400 can act as a peer machine in peer-to-peer (or other distributed) network environments. The machine 400 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 400. Further, while only a single machine 400 is illustrated, the term “machine” shall also be taken to include any collection of machines that individually or jointly execute a set (or multiple sets) of instructions to perform any one or more of the methodologies discussed herein. Example machine (e.g., computer system) 400 can include a processor 402 (e.g., a central processing unit (CPU), a graphics processing unit (GPU) or both), a main memory 404 and a static memory 406, some or all of which can communicate with each other via a bus 408. The machine 400 can further include a display unit 410, an alphanumeric input device 412 (e.g., a keyboard), and a user interface (UI) navigation device 411 (e.g., a mouse). In an example, the display unit 410, input device 412 and UI navigation device 414 can be a touch screen display. The machine 400 can additionally include a storage device (e.g., drive unit) 416, a signal generation device 418 (e.g., a speaker), a network interface device 420, and one or more sensors 421, such as a global positioning system (GPS) sensor, compass, accelerometer, or other sensor.

The storage device 416 can include a machine readable medium 422 on which is stored one or more sets of data structures or instructions 424 (e.g., software) embodying or utilized by any one or more of the methodologies or functions described herein. The instructions 424 can also reside, completely or at least partially, within the main memory 404, within static memory 406, or within the processor 402 during execution thereof by the machine 400. In an example, one or any combination of the processor 402, the main memory 404, the static memory 406, or the storage device 416 can constitute machine readable media.

While the machine readable medium 422 is illustrated as a single medium, the term “machine readable medium” can include a single medium or multiple media (e.g., a centralized or distributed database, and/or associated caches and servers) that configured to store the one or more instructions 424. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine-readable media can include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 424 can further be transmitted or received over a communications network 426 using a transmission medium via the network interface device 420 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

This disclosure introduces a novel wearable optical spectroscopy framework designed for low-cost, non-invasive monitoring of melanin, erythema, and skin tone. This disclosure utilizes an off-the-shelf multi-spectrum wearable device available in various configurations to enable real-time, personalized assessments across diverse skin conditions and skin tones. This disclosure will assess the performance of the disclosed algorithm against a state-of-the-art colorimeter in a comprehensive user study involving a diverse group of 77 subjects, demonstrating a normalized mean absolute error (NMAE) of 5.33% (melanin) and 4.18% (erythema), and ΔE values less than 2.5 for CIE LAB measurements. Furthermore, this disclosure will present an algorithm that utilizes outputs to correct for pulse oximeter inaccuracies typically found in those with darker skin pigmentation, resulting in an up to 75% decrease in mean absolute error (MAE) in hypoxic readings across skin tones relative to arterial blood measurements.

Our findings highlight potential for a short and long-term skin monitoring tool as a significant enhancement to existing wearable devices, particularly in improving the accuracy of pulse oximeter readings across different skin tones.

In the fields of dermatology and cosmetic science, precise analysis of skin characteristics serves as a key measure in diagnosing various skin conditions and tailoring cosmetic and medical interventions. Three of the most significant aspects in this analysis are melanin content, erythema level, and skin-tone, all of which not only characterize the overall appearance of the skin, but its physiological response to environmental factors and susceptibility to various dermatological conditions [1, 20, 58, 92]. These skin characteristics are also crucial factors in the performance of optical sensors used in wearable health monitoring devices, as they can significantly affect the accuracy of measurements like blood oxygen saturation [8, 69] and heart rate [44] by altering the absorption and scattering of light through tissue. Furthermore, these characteristics are not static; they can fluctuate significantly due to factors such as exposure to sunlight, hormonal changes, and/or skin injury [92], thus requiring the need for continuous monitoring or frequent periodic assessment to account for these dynamic changes.

Traditional practices for assessing these markers of skin have ranged from subjective visual assessments, such as the Fitzpatrick [28] or Monk [54] Skin Tone Scales, to more objective yet often invasive or sparse quantitative methods, such as biopsies [62] or portable spectrometers in the form of skin-tone pens [2, 12, 15, 19, 82]. These conventional methods, while valuable in specific contexts, each present notable limitations: subjective scales like the Fitzpatrick and Monk assessments inherently suffer from observer variability and lack quantitative precision; portable spectrometers, though objective, are typically expensive, require frequent calibration, and only provide discrete measurements rather than continuous monitoring; and biopsies, while providing detailed cellular information, are invasive procedures that cannot be performed frequently enough for ongoing monitoring of dynamic skin changes.

The advent of wearable technology offers a promising solution for non-invasive monitoring of health parameters. Within this context, wearable optical spectroscopy is a particularly useful approach for objective, long-term skin-tone analysis. These devices present a cost-effective alternative to the aforementioned skin-tone pens, which cost several thousand dollars. Current wearable devices already use optical spectroscopy to measure biomarkers such as heart rate and blood oxygen, which can be extended to potentially measure skin composition. Developing a wearable device capable of measuring melanin, erythema, and skin-tone continuously and non-invasively can be beneficial in several ways:
(1) Addressing the pulse oximeter problem—the ability to measure melanin and erythema in a wearable form-factor can improve the accuracy of pulse oximeters, which often show biased readings in individuals with darker skin [27, 36, 43, 71, 79], as well as enhance the reliability of other optical biosensors that operate under similar principles.
(2) Diverse Skin-Tone Inclusivity—incorporating comprehensive data on melanin and skin-tone across diverse skin-tones can enhance representation and fairness, especially in AI model deployment in healthcare or remote patient monitoring/recruitment.
(3) Personalized Skin Care—understanding individual skin composition can aid in personalized treatments, especially in dermatology where personal skin properties can have dramatic influences on treatment effectiveness.
(4) Sun Exposure Management—monitoring changes in melanin and erythema content can provide personalized recommendations for sun protection, potentially reducing the risk of skin cancer by allowing for timely and tailored interventions based on an individual's unique skin responses to UV exposure.

In this paper, this disclosure will address the following research questions:
RQ1: How can this disclosure non-invasively measure melanin, erythema, and skin-tone content accurately using wearable optical spectroscopy?
RQ2: How can this disclosure use these skin measures to correct for inaccuracies seen in pulse oximetry measurements in darker skin-tones?

This disclosure will introduce a novel optical spectroscopic framework that enables non-invasive, continuous analysis and reporting of melanin, erythema, and overall skin-tone. The framework of this disclosure uses a commercially available device that utilizes reflectance spectroscopy with wavelengths across the visible spectrum in multiple wearable form factors. The device leverages a multi-spectral sensor paradigm to enhance visible light resolution beyond that of current commercial wearables, allowing for greater data density. This disclosure will leverage this data to differentiate between subtle changes in skin properties and make a quantitative assessment of the underlying structure of the skin.

This disclosure will develop an algorithm that processes multi-spectral signals obtained from reflectance spectroscopy and returns melanin, erythema, and skin-tone values. The algorithm processes raw three-dimensional spectral data and employs a machine learning model to optically differentiate between skin constituents across various skin-tones. Our approach achieves normalized mean absolute error (NMAE) values of 5.33% for melanin and 4.18% for erythema, with ΔE values below 2.5 for CIE L*, a*, and b* measurements-a standardized color space used for skin-tone where L* indicates lightness/darkness, a* represents green-red, and b* represents blue-yellow-respectively, across skin-tones, with consistent results regardless of overall skin-tone and measurement location. This disclosure will evaluate our algorithm on its accuracy, mean absolute error, and generalizability relative to a skin-tone pen used and trusted in dermatological settings.

Furthermore, this disclosure developed an algorithm tested against an open-source database that incorporates skin-tone measurements into blood oxygen measurements to correct inaccuracies found in pulse oximeter measurements, which is amplified in subjects with darker skin pigmentation. Overall, this disclosure will demonstrate up to a 75% decrease in mean absolute error (MAE) and a decrease in overall variability across skin-tones simply by incorporating these skin-tone values in blood oxygen prediction.

Our contributions can be summarized as follows:
(1) An end-to-end framework that employs commercially available technology for non-invasive, continuous skin-tone monitoring.
(2) A model for transforming wearable multi-spectrum data to melanin, erythema, and skin-tone measurements.
(3) A comprehensive user study demonstrating the accuracy of this system across a variety of skin-tones and body locations.
(4) An algorithm to correct pulse oximeter inaccuracies in darker skin pigmentation by incorporating skin measurement in blood oxygen calculation.

The remainder of this work is structured as follows: This disclosure provides a background of melanin, erythema, and skin-tone, and the methods that have been used to attempt to quantify them. This disclosure details the framework of this disclosure, specifically the off-the-shelf device that is used for system set-up, the gold standard device that is used for comparison, and the algorithm developed for reporting of melanin, erythema, and skin-tone, as well as correcting for pulse oximeters. This disclosure will evaluate our device against the gold standard measurements in a user study and evaluate our algorithm to correct pulse oximeter inaccuracies in dark skin participants. Lastly, this disclosure will discuss results and plans for future work.

There has been substantial fundamental research on melanin and overall skin-tone constituents. Melanin, the pigment primarily responsible for skin color, plays a critical role not only in cosmetic appearance but also in protecting the skin from UV radiation [45, 59, 94]. Erythema, often manifested as redness of the skin, can indicate inflammation, allergic reactions, or other skin conditions [76]. Understanding the relation of these two skin constituents, their interaction with one another, and traditional measurement practices for these biomarkers is important in accurately assessing skin health. This disclosure will begin this section with an overview of melanin and erythema, two components of skin-tone that dramatically impact its visual appearance as well as its response to environmental and clinical factors. Then, this disclosure will discuss the traditional visual approaches of skin-tone quantification and classification. Lastly, This disclosure will discuss quantitative methods that have been developed to enhance these approaches.

The visual appearance of skin is largely dictated by its chromophores, a term coined to describe specific molecules that absorb light radiation and emit color as a result. The main chromophores affecting skin color are melanin, which imparts a range of shades from brown to black, and hemoglobin, which causes erythema or redness; minor chromophores such as DNA and certain proteins are impactful in absorbing radiation, specifically in the ultraviolet (UV) spectrum, and thus play a more dominant role in skin protection, rather than altering visible color [92]. However, upstream effects can be demonstrable in cases such as DNA damage due to excessive UV exposure, where variations in melanin and/or hemoglobin can appear on the skin surface as a result. Thus, the interplay of these chromophores not only determines skin color variability among individuals but can also provide insight on the skin's physiological state [58, 92, 94].

Melanin is a complex polymer derived from the amino acid tyrosine and plays a pivotal role in determining skin color, as well as acting as a natural sunscreen by absorbing potentially harmful UV radiation. Analysis of melanin content is crucial for the assessment of various dermatological conditions which may manifest through abnormal melanin levels [45, 55, 59, 63, 94]. An increase in melanin might suggest conditions like hyperpigmentation or lentigo, whereas a decrease could be indicative of conditions such as vitiligo or hypopigmentation. Furthermore, melanin content can directly impact susceptibility to sunburn, as well as skin cancer [55, 94]. Skin melanin can be categorized into two basic types: eumelanin, which is visually black-brown, and pheomelanin, which is visually red-yellow. The ratio of these melanin derivatives directly determines skin composition, including how the skin may appear visually; pheomelanin, for example, is found at high-levels in those with pale skin and red or fair hair, while eumelanin is found at high-levels in those with darker skin pigmentation [55, 68]. Erythema level is another significant constituent of skin-tone that directly influences the visual appearance of skin. Erythema is a common dermatological condition characterized by redness or rash on the skin resulting from capillary dilation and increased blood flow. The characteristic redness of erythema can be attributed to an increased concentration of hemoglobin in the affected area. Erythema is particularly important in the detection of early signs of skin wounds or infections and is present in skin that is affected by conditions that may be considered cosmetic, such as acne. It can also serve as an indicator of systemic diseases or reactions to medications. However, assessing erythema can be challenging, especially in those with darker skin pigmentation, potentially leading to underdiagnosis or delayed treatment of skin conditions [1, 76, 92].

Clinical evaluation of skin must involve analysis of color and composition, among other factors; both melanin and erythema are critical in these analyses [76]. However, the influence of melanin on clinical measurements has historically complicated clinical assessment. Erythema, for example, has largely been more difficult to assess in darker skin-tones due to melanin's dominant effect on skin color [18, 22, 32]. Healthcare provider difficulty in assessing erythema in darker skin tones has been implicated in health disparities associated with conditions such as pressure injuries, sexual assault injuries, Lyme disease, and the identification of limb ischemia among racial or ethnic groups with dark skin tones [6, 29, 50, 51, 61, 65-67, 75]. Further, the influence of melanin on these assessments extends to other clinical measurements, such as the potential for inaccurate pulse oximetry readings, where melanin's absorption properties can affect device accuracy. There has been substantial research on the impact of melanin on pulse oximeter measurements, which has been directly linked to negative health outcomes in those with darker skin pigmentation [25, 26, 35, 38, 40, 42, 57, 70, 73, 78, 84]. The under-detection of hypoxemia as a result has led to differences in treatment decisions, including administration of supplemental oxygen, intravenous dexamethasone, and hospital admission, which was particularly evident during the COVID-19 pandemic [25, 26, 78]. Furthermore, these differences may also extend towards other optically obtained biomarkers such as heart rate, where it is still unclear whether there is a racial bias in reporting of heart rate in wrist-worn wearables [5, 8, 13, 14, 44, 69]. These challenges have necessitated the need for tools and devices to quantify and classify skin-tone and skin composition. Dermatological methods for classification of skin-tone have ranged from subjective assessment to quantitative approaches of skin composition. Traditionally, visual assessment has been a critical component of these practices using scales such as the Fitzpatrick [28], Von Luschan [87], Pantone [60], and the recently developed Monk Skin Tone Scales [54], all of which utilize visual and descriptive criteria to categorize skin color and its potential response to UV exposure. These systems are particularly focused on classifying skin-tones, which is mainly dictated by melanin; Clinical Erythema Assessment (CEA) [81] is a descriptive scale that is used to categorize erythema severity. While these systems differ in the depth and scope of their classifications, they share a common limitation in providing an objective, unbiased classification of skin composition. The embodiments of this disclosure offer quantitative measurement of melanin, erythema, and skin-tone, without the need for subjective classification.

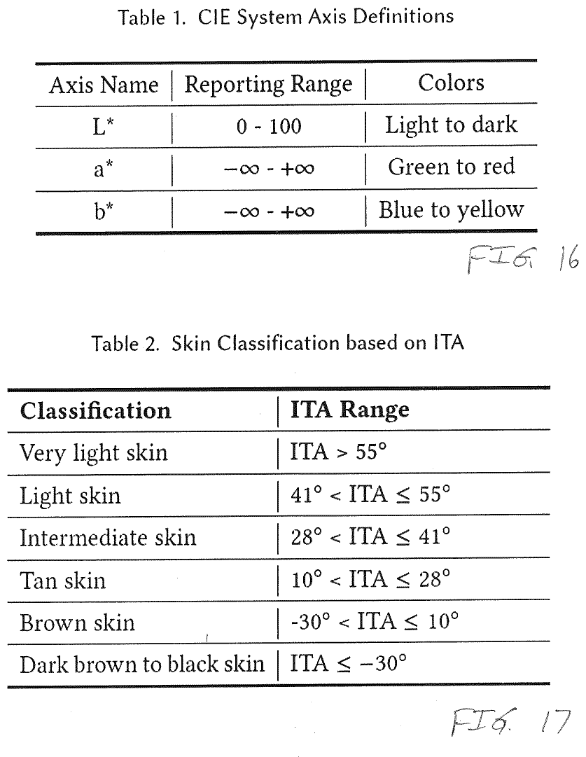

Given the optical properties of melanin, reflectance spectroscopy has been a prominent method for its study. There has been substantial research dedicated to examining the reflectance spectra of chromophores, particularly melanin, in human skin. Many of these studies have reported strong absorption characteristics in the approximate spectral range of 600-800 nm by measuring the skin's reflectance in various conditions [46, 64, 93, 94]. Similarly, the spectral response of erythema, primarily influenced by hemoglobin content, has been evaluated using similar methods. While traditional pulse oximetry uses red (660 nm) and infrared (940 nm) wavelengths to measure blood oxygenation [10, 56, 83]-wavelengths chosen for their tissue penetration depth and relative isosbestic properties-the assessment of erythema at the skin level focuses on different spectral regions. Erythema is typically characterized in the visible spectrum, particularly between 500 nm-700 nm with strong absorption in the green region [24, 77]. Furthermore, the spectral response of both melanin and hemoglobin is dependent on the location of measurement [64, 93], as different body sites vary in their blood perfusion and tissue composition, thereby necessitating careful consideration of measurement sites to ensure accurate and consistent assessment. See Table 1 at FIG. 16

Recently, colorimeters have been developed as a quantitative measure of skin-tone using optical spectroscopy by examining remittance spectra across the visible spectrum. Current commercial colorimeters are handheld devices that are used to objectively measure skin color using a three-dimensional color space following the Commission Internationale de l'Eclairage (CIE) system [90]. This system is used to represent colors in a way that is more aligned with human vision, as opposed to the traditional RGB or CMYK systems used in digital or print mediums. The CIE categorizes its axes as shown in Table 1. While a* and b* are technically unbound, they are often clamped to the range of −128 to 127 for use with integer code values. In addition to these values, colorimeters also often report the individual typology angle (ITA). ITA is a measure used to quantify skin color using the CIE scale, specifically using the L* and b* components. The formula for calculating ITA is Formula (1):

ITA=arctan ((L*−50)/b*)×180/pi

The result, expressed in degrees, helps categorize skin into various typological groups. Skin is classified based on ITA measurement using the ranges specified in Table 2 of FIG. 17. ITA provides a quantitative measure of skin-tone that is classified into interpretable groups, as opposed to more subjective approaches that are typically used in this context such as the Fitzpatrick [28] skin-tone scale, thus making it the preferred approach to quantify skin-tone if available. This technique is particularly useful in cosmetic science and dermatology to analyze skin characteristics and potential cosmetic products based on skin undertones [16, 41, 48]. See Table 2 in FIG. 17. Colorimeters that utilize the CIE system can accurately measure skin color independent of the actual device that is being used for measurement. Since melanin and erythema directly impact red and green measurements, many colorimeters also report an erythema and melanin value as well. However, a confounding factor in the analysis of these two skin constituents is that their absorption spectra overlap (600-800 nm range). Thus, implementation across devices for the extraction of these two biomarkers can differ, especially in the wavelengths that are utilized to infer the measurements. A summary of colorimeters and their analysis techniques is shown below in Table 3. All of these devices utilize reflectance spectroscopy, but may or may not report CIE LAB, melanin, and erythema levels altogether [41, 48]. See Table 3 in FIG. 18.

As previously mentioned, accurate and comprehensive skin analysis requires examining two key components: skin physiology (specifically melanin and erythema levels) and overall skin color. While melanin and erythema are fundamental physiological markers, skin color can vary independently due to various factors including diet [91], environmental conditions [21], and the use of cosmetics. This makes measuring both skin color and the underlying physiology essential for a complete assessment. Of the colorimeters available on the market today, only two, the Cortex DSM IV [15] and the Delfin SkinColorCatch [82], report melanin, erythema, and skin-color measurements. While these colorimeters offer valuable capabilities for clinical dermatology applications, they face several practical limitations: high cost (often several thousand dollars, with prices typically restricted to institutional buyers), bulky clinical form factors, requirements for periodic calibration, and limited data collection frequency. In contrast, embodiments of this disclosure provide a cost-effective solution for continuous monitoring of melanin, erythema, and CIE LAB values, designed for integration across various wearable formats.

To date, there has been limited work on sensors and devices to monitor skin condition in a wearable or low-cost form-factor. Sundroid [23] was recently developed as a wearable prototype system that measures incidental UV radiation using a body-worn sensing unit to alert users of harmful sunlight exposure. The system achieves this by directly measuring sunlight, rather than skin properties, and is thus not directly fixated directly on the skin but can be attached to the user's clothing. Several commercial devices use a similar principle, such as My UV Patch by La Roche-Posay [47], SunIndex [80], and LogicInk [49], which directly track UV exposure. Other devices have utilized imaging techniques to quantify skin lesions and potential dermatological conditions, such as the DermLite DL5 [17], MoleScope [53], and SkinVision [74]. Recently, a low-cost skin spectrum measurement device was developed which provides melanin, erythema, and skin-tone content using optical spectroscopy in a small 3D printed housing structure [39]. The device utilizes a deep neural network model, along with an accompanying mobile application, to provide insights on these parameters. However, the device is not designed for continuous use, as it needs to be held against the skin. Furthermore, discussion on the model, considering the small subject sample size for the device (16 subjects) and the training model used, is limited especially pertaining to efforts used to prevent potential overfitting. There is also no discussion regarding the diversity or skin-tone classification of the recruited subject group. Overall, none of these devices or applications provide detection of melanin, erythema, or skin-tone, and/or are limited to discrete measurements rather than continuous data collection. This disclosure offers a flexible solution that enables real-time, continuous analysis of skin composition directly and is developed on a diverse group of skin-tones, expanding upon commercial skin monitoring solutions and colorimeters by enabling these analyses in daily routine.

In this section, this disclosure will discuss the components utilized to develop the framework. This disclosure will first describe the off-the-shelf device that this disclosure utilized to gather data for algorithm development. Next, this disclosure will describe the gold standard device that this disclosure utilized as reference measurement. Lastly, this disclosure will discuss the algorithmic pipeline developed to report melanin, erythema, and skin-tone using the framework, and the methods used to correct pulse oximeter readings using skin-tone measures. FIG. 7 and FIG. 8 demonstrate an overall framework architecture for embodiments of this disclosure. Each step is elaborated upon in subsequent sections.

This disclosure utilized Lumos [89], an off-the-shelf wearable device, to gather optical spectroscopy data. Lumos is a multi-spectrum device that utilizes light-emitting diodes (LEDs) and photodiodes (PDs) that cover the entire visible spectrum, ranging from 400 nm to 1000 nm, allowing us to fully examine the spectral properties of the skin in a radiation range that is safe. The device is designed to be versatile and is available in multiple form-factors, which can measure skin content on any location in the body. Lumos utilizes off-the-shelf LEDs and PD arrays on a custom PCB, with components totaling less than $50.

Lumos contains 9 LEDs and 10 PDs across the visible spectrum to optimize spectral resolution. Each LED operates within a lumen range of 104-114 and covers wavelengths from 415 nm to 910 nm. The PD channel can detect wavelengths from 350 nm to 1000 nm, operating at a sampling rate of 0.3 Hz. The operable spectral response of the LEDs and PDs for the Lumos device can be found in FIGS. 3a and 3b below, respectively.

While these components can be adjusted to capture wavelengths beyond this range, they are particularly suited for our current application given the specific absorption characteristics of melanin and erythema. Regardless of configuration, Lumos captures spectral data in reflectance mode. It cycles through each LED, sequentially activating them and recording the corresponding PD responses. This cycle repeats, allowing a complete sweep of all 9 LEDs and corresponding 10 PDs in approximately 3 seconds. This systematic process ensures that This disclosure will collect thorough and precise spectral data consistently.

This disclosure utilized the Cortex DSM-IV as a comparative gold standard against Lumos output data. This disclosure did not use a visual scale such as the Fitzpatrick scale to avoid subjective bias in our model. Our goal was to ensure that reporting was as comprehensive as possible by using a device capable of accurately reporting melanin, erythema, and CIE LAB values. As mentioned previously, the current colorimeters on the market that meet these criteria are the Delfin SkinColorCatch [82] and Cortex DSM-IV [15]. Both of these devices use similar spectral ranges to report these parameters as shown in Table 3 of FIG. 18.

The comparative literature on these devices is limited but informative. Van der Wal et al. [85] compared the performance of these colorimeters against several visual scales and found them both to provide reliable color data on skin and scars with a single measurement. Baquie et al. [4] compared the SkinColorCatch with the Mexameter, and found the SkinColorCatch to be far more reliable and robust over a range of melanin and erythema values.

To our knowledge, there has been no direct comparison of the SkinColorCatch against the DSM-IV. While both the DSM-IV and SkinColorCatch offer comparable technical capabilities, several factors influenced our selection of the DSM-IV colorimeter:
(1) Cost effectiveness: the DSM-IV presents a relatively more economical option compared to the SkinColorCatch without compromising measurement quality.
(2) Operational efficiency: the DSM-IV features built-in calibration protocols, whereas the SkinColorCatch requires intermittent in-lab calibration.
(3) Availability: With the discontinuation of the DSM-II and DSM-III models by Cortex Technology, the DSM-IV represents the only currently supported version of this colorimeter.
(4) Technical advancements: as an upgrade to the discontinued DSM-II and DSM-III, the DSM-IV incorporates significant improvements, including but not limited to enhanced spectral sensing, improved software algorithms, and better reproducibility.

These advantages, particularly the DSM-IV's enhanced sensor technology and updated calibration protocols, address the limitations noted in earlier DSM-II studies while matching the capabilities of the more expensive SkinColorCatch. Overall, these features make the DSM-IV an ideal choice for benchmarking Lumos performance in quantitative skin analysis.

Lumos provides raw spectral data in the form of analog-to-digital-converter (ADC) counts for each photodiode in each cycle of LED flashed during its operation. Thus, for each cycle of Lumos measurements having 9 LEDs and 10 PDs, there are a total of 90 PD responses, where each value is the LED that is turned on at that moment in time, with its corresponding PD. Correspondingly, this data can become very dense as it is operating on multiple scales (LED, PD, ADC counts, time). To visualize and analyze this data, this disclosure utilized SpectraVue [37], an open-source platform that supports visualization of multi-spectrum signals. This platform is especially helpful to gain an underlying intuition for any patterns or trends that can be gathered heuristically. Utilizing both SpectraVue and techniques used in traditional photoplesmography, This disclosure will developed the following features:
(1) Mean value of PD responses-calculate the mean value of each PD response over the course of the measurement. (2) Direct current (DC) content of each PD response—this feature was calculated based on the assumption that underlying skin properties, such as melanin, erythema, or skin-tone, do not change instantaneously during a relatively short sampling period (<1 minute). Therefore, any alternating current (AC) components detected in the spectral response are attributed to noise or biomarkers not targeted in this study. (3) arctan of each PD response—a domain specific technique that is used to calculate ITA; the resulting angle can be interpreted as a measure of the relative contribution of different spectral components, similar to how ITA represents the relationship between color scales in skin-tone measurement.
(4) PD ratios with one another—in pulse oximetry, blood oxygenation is traditionally determined by calculating the ratio of absorption between the infrared and red light responses of the body. This method, known as the ratio-of-ratios (ROR), involves comparing the AC and DC components of both waveforms relative to each other [10, 56, 83]. This disclosure will have extended this technique to a multispectral context by calculating ratios between various responses from different LED and PD combinations. This disclosure will have also taken ratios of more advanced features (such as DC content of various PD responses) as a regularization technique to maintain feature monotonicity while reducing the impact of extreme values.

To extract the baseline skin content levels for feature selection, This disclosure will high-pass filter all LED and PD combinations during a measurement period using a 2nd-order Butterworth filter with a 0.1 Hz cutoff frequency. In this way, this disclosure will effectively extract the DC component of the underlying signal, which This disclosure will assume to be indicative of skin content.

This methodology also helps remove any motion artifact that may occur during data collection. Following this filtering, as well as calculating the arctan of each LED and PD response, This disclosure will then took ratios of all features with each other as an additional regularization measure.

This disclosure tested several supervised learning algorithms by utilizing the aforementioned multivariate features to predict each of the desired outcome variables, namely melanin, erythema, L*, a*, and b* values. Since ITA is directly calculated using L* and b* values, This disclosure will did not create a model for its prediction. This disclosure will aimed to develop a generalizable and robust model by training several different linear and tree-based regression algorithms against each target variable, including linear regression, Ridge regression, RandomForest, XGBoost, LightGBM, and CatBoost models.

The total feature set was reduced to 10 features for melanin and erythema, and 50 features for CIE LAB values, using recursive feature elimination (RFE) with mean absolute error (MAE) as the optimization criterion. During RFE, the native feature importance metric of each model was used to rank features: coefficient magnitude for Lasso and Ridge regression, variance reduction for RandomForest, ExtraTrees, and GradientBoosting, gain for XGBoost, and loss-function-based importance for CatBoost and LightGBM. Model hyperparameters were tuned using a grid search and selected based on achieving the optimal balance between accuracy and model complexity. Models were validated using both 5-fold cross-validation with subject stratification and Leave-One-Out Cross-Validation (LOOCV) of Table 4 at FIG. 19 to prevent subject data leakage between training and validation sets while ensuring robustness of our results across different validation strategies.

Model performance was evaluated using multiple metrics: for melanin and erythema predictions, This disclosure will used R2, normalized mean absolute error (NMAE), and normalized root mean squared error (NRMSE). These normalized metrics enable meaningful comparisons for melanin and erythema, whose values are abstract. For CIE LAB color predictions, This disclosure will used Delta E (ΔE) the industry standard for color difference measurements in LAB space. ΔE represents the Euclidean distance between predicted and true colors, as shown in Eq 2. Formula:

Delta E=Square Root ((Delta L*)²+(Delta a*)²+(Delta b*)²)

Unlike individual color component errors, ΔE provides a perceptually relevant measure of total color difference that aligns with human visual perception. In clinical and dermatological applications, ΔE values less than 2-3 are considered acceptable as they represent color differences that are difficult to distinguish without careful observation. Full evaluation of model performance using these metrics can be found in this disclosure.

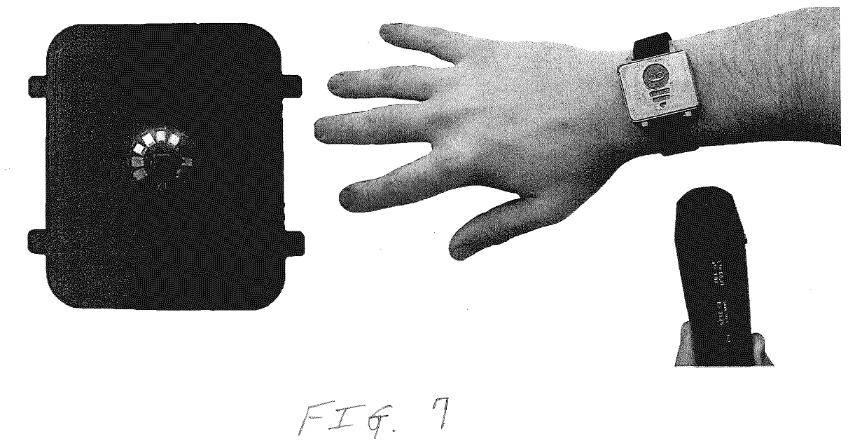

This disclosure will evaluated the feasibility of correcting pulse oximeter measurement inaccuracies in darker skin tones using the OpenOximetry dataset from PhysioNet [30, 33]. This centralized repository contains open-source pulse oximetry data, reference arterial line measurements, and spectrophotometer measures, including melanin, erythema, and LAB values, across a variety of skin-tones, measurement locations, pulse oximeter devices, and spectrophotometers. The repository classifies pulse oximeters based on their performance across different skin tones, enabling systematic evaluation of device accuracy among demographic groups. Within the dataset, each patient participates in multiple encounters where various pulse oximeter devices are tested against arterial blood oxygen measurements, while spectrophotometer readings are simultaneously collected across different anatomical sites. This disclosure will analyzed data across this repository to inspect differences in blood oxygen measurements obtained through pulse oximeters relative to arterial line measurements in various skin-tones to determine whether blood oxygen measurements can be corrected by utilizing skin-tone information. A detailed process flow diagram for the developed pulse oximetry bias correction algorithm can be found in FIG. 10. The pipeline processes raw data collected from blood gas measurements (SO2), pulse oximeter readings (SpO2), and spectrophotometer values, comprising 99 patients across 238 encounters. Given that encounters contain spectrophotometer measurements from multiple anatomical locations, This disclosure will isolated fingernail measurements to align with the exclusive use of finger pulse oximeters in the repository. This disclosure will then implement a filtering criterion to exclude cases where the difference between SO2 and SpO2 exceeded 3%. This threshold was selected for two primary reasons: the OpenOximetry database's pulse oximeter accuracy classification is not yet reflected in the raw relational database and thus non-compliant devices cannot be determined inherently, and FDA guidance stipulates pulse oximeter accuracy should fall within 3% of ground-truth measurements [31]. This refinement yielded a final dataset of 96 patients with 221 corresponding encounters. To minimize the influence of raw saturation values on model predictions, This disclosure will engineered relational features through various polynomial and trigonometric combinations of L*, A*, and B* values. These engineered features, along with a single device identifier, serve as inputs to a CatBoost regressor model which employs leave-one-subject-out cross validation to prevent data leakage. The unique device identifier is used as different devices have different calibration curves for blood oxygen calculations, which can affect their bias for the same participant. Model performance is evaluated based on mean absolute error improvement relative to the baseline oximeter prediction as well as bias correction differences across skin tone groups; these results are presented herein.

In this section, this disclosure will describe the methods used to verify outputs of the embodiments herein relative to the DSM-IV. First, this disclosure will outline the experimental procedure used to gather across skin-tone data across a diverse group of subjects. Next, this disclosure will evaluate our model's accuracy and generalizability across these skin-tones against the DSM-IV output. Lastly, this disclosure will assess the performance of utilizing skin-tone measurements to correct for pulse oximeter measurements especially found in those with darker skin pigmentation.

This disclosure will evaluated the accuracy of the framework discussed herein in an IRB-approved (IRB-HSR #301300), 77 person user study. Participants were not directly recruited based on their skin-tone, race, gender, or ethnicity; recruitment was conducted broadly to reach a general population without specific targeting. Since skin content values can depend on location of measurement, This disclosure recorded both Lumos measurements and DSM-IV measurements on 3 sites of the body: the palmar side of the fingertip, the dorsal side of the wrist, and the shoulder. This variety in measurement locations allows the device to operate effectively in a location-agnostic manner. Lumos measurements were collected at each site for approximately 60 seconds, with a subsequent DSM-IV measurement at the same site. Each measurement was repeated 5 times per location, totaling approximately 1155 observations.

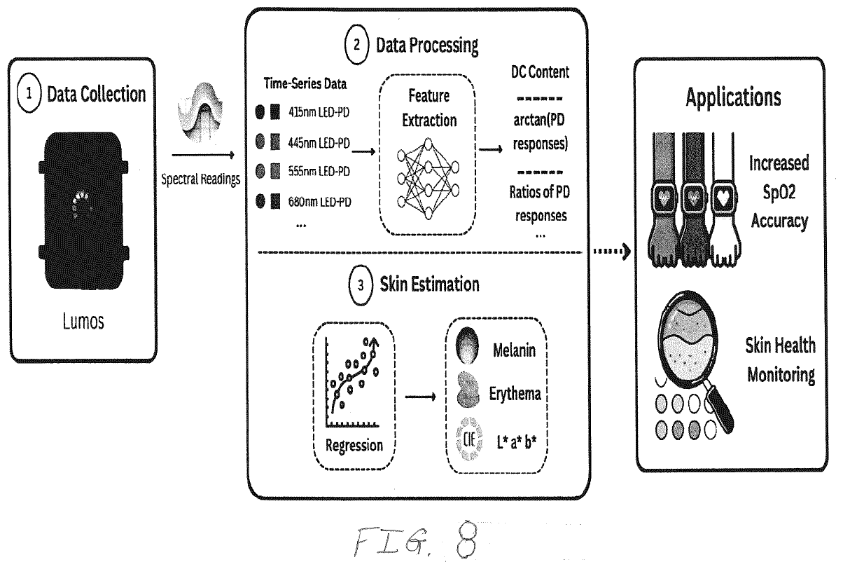

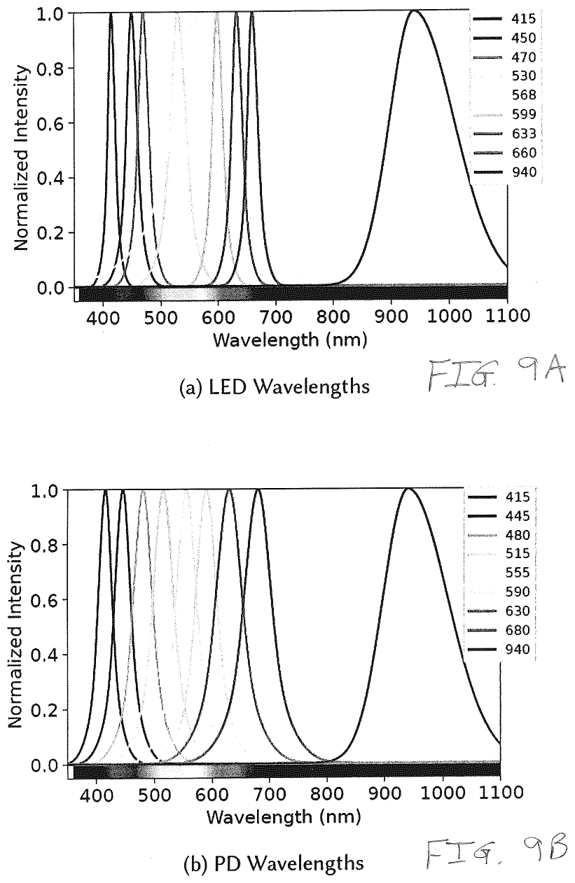

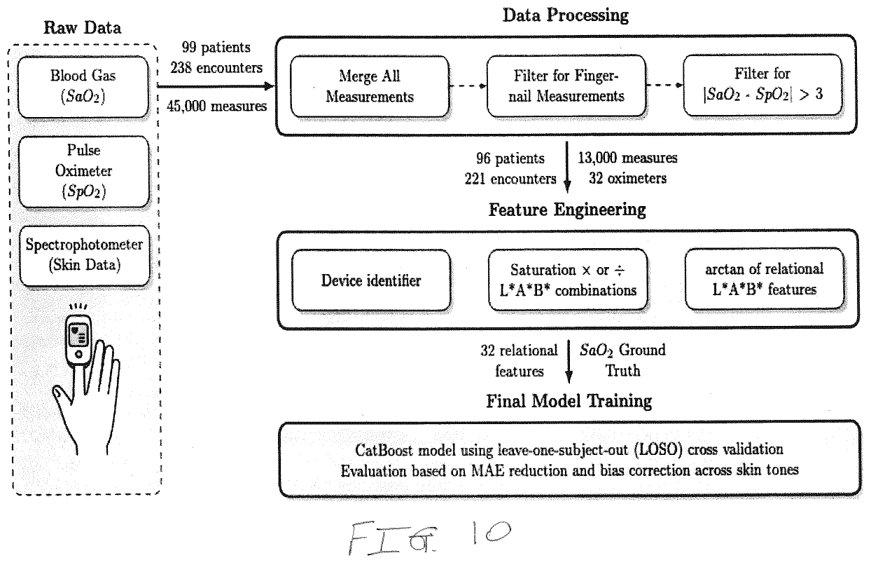

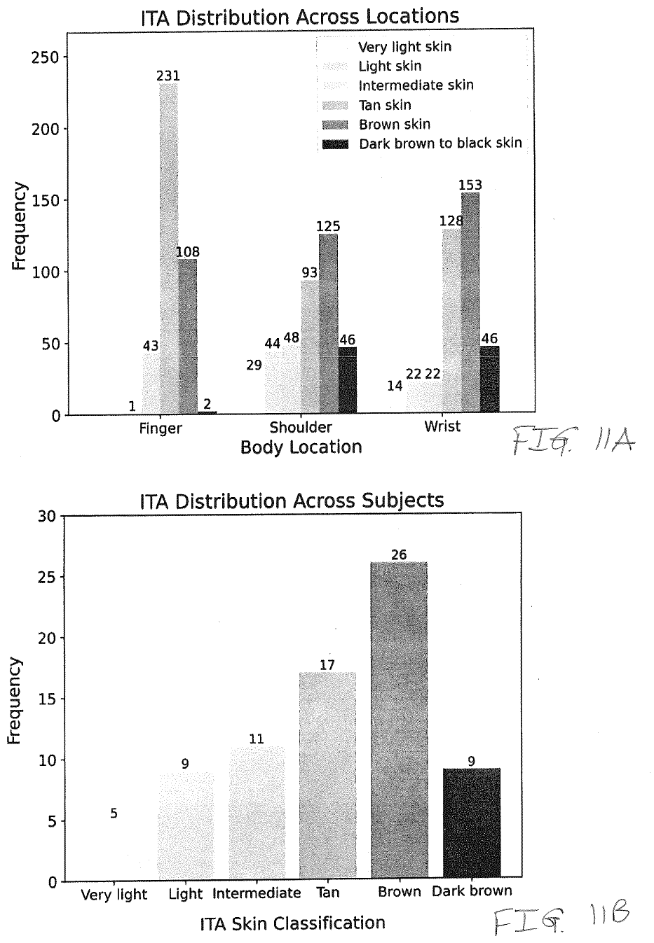

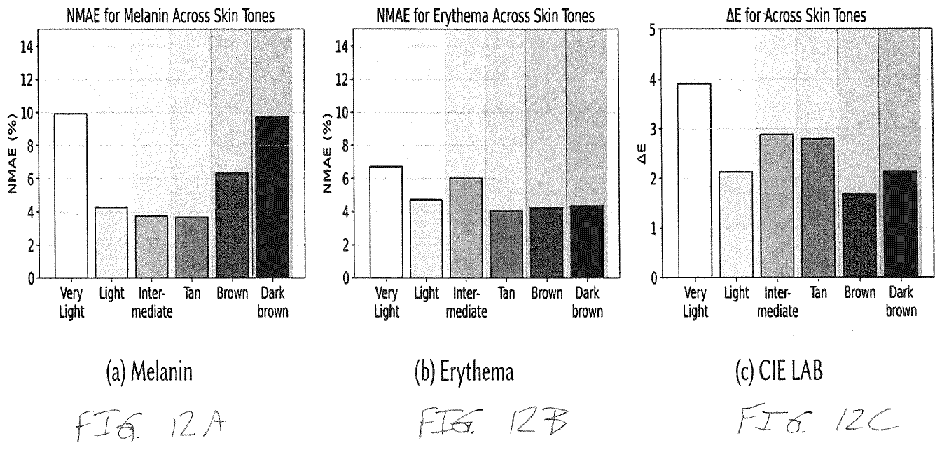

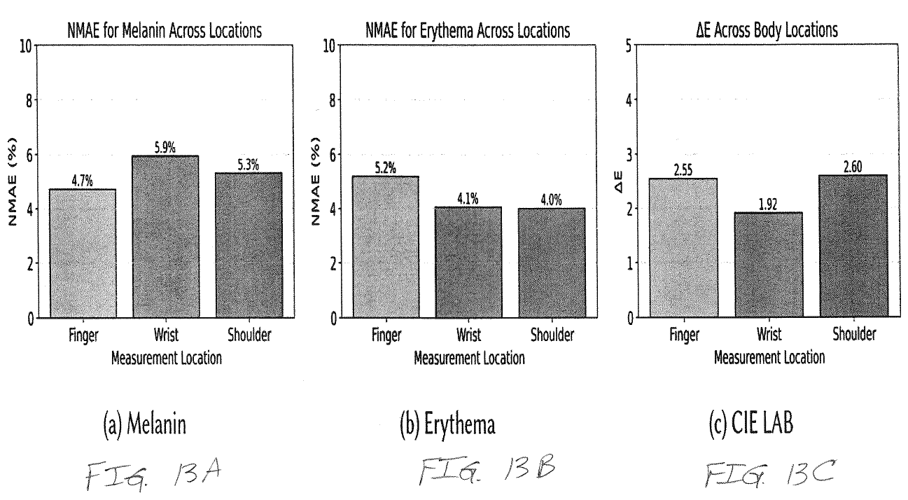

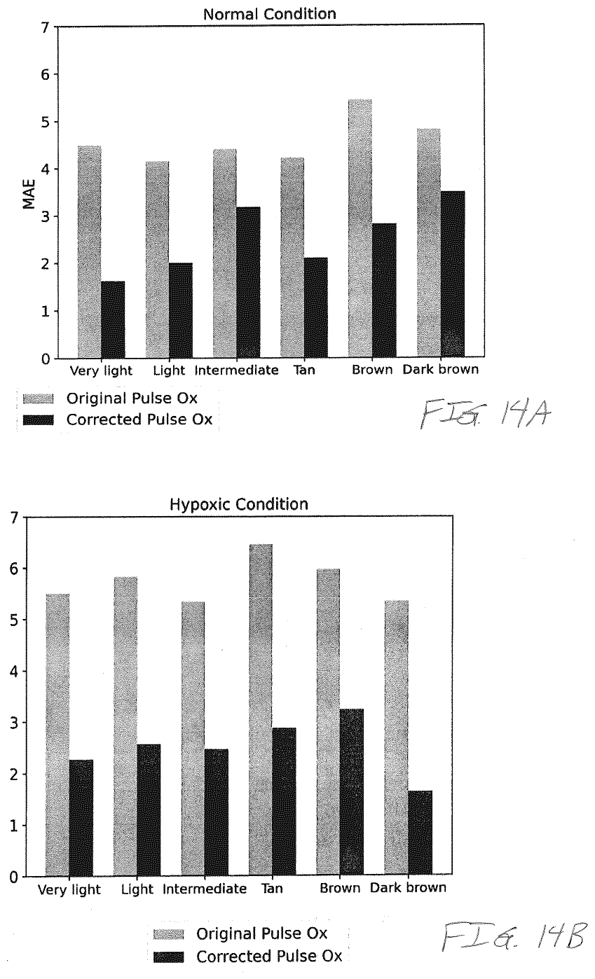

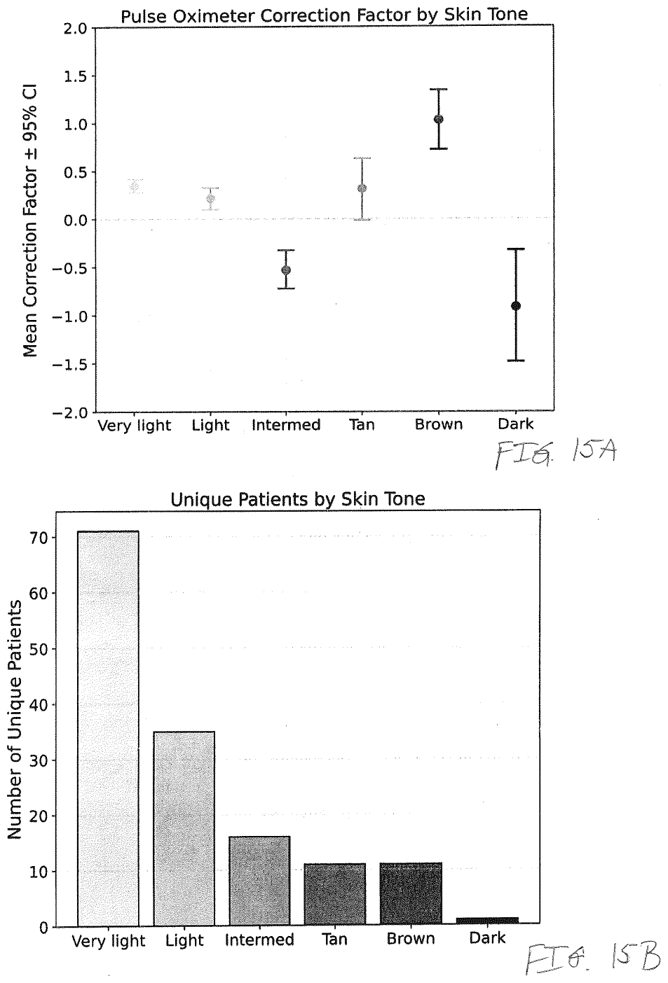

A histogram of the ITA classification of study participants, as well as the ITA classifications of each body location can be found in FIGS. 11A and 11B. As shown in FIGS. 11A and 11B, which represent the frequency distribution of ITA measurements across all three body locations (fingertip, wrist, and shoulder) and the distribution of the first shoulder measurement of each subject alone, respectively, the ITA classification distributions exhibit notably different patterns when comparing measurements across all body locations versus a single shoulder measurement.

The shoulder measurements (FIG. 11B) were used as the primary indicator of subjects'natural skin tone, as this measurement location typically experiences minimal environmental exposure compared to the fingertip or wrist, making it less susceptible to temporary alterations like tanning. While both distributions approximately follow a normal distribution centered around the tan and brown categories, FIG. 11A demonstrates how measuring different body locations can significantly skew the apparent distribution of skin tones, with a markedly higher frequency of tan classifications compared to the shoulder-specific measurements, especially with regards to finger measurements. This finding suggests that finger measurements naturally cluster toward intermediate skin tones, regardless of the subject's overall skin-tone.

In this section this disclosure will present our results for melanin, erythema, and CIE LAB prediction using both LOOCV and 5-fold cross validation across several regression models. This disclosure will contextualize these results across measurement locations, as well as skin-tones, and provide important features for each predictor.

LOOCV Results. FIG. 19. The results of the five highest performing models using leave-one-out-cross-validation (LOOCV) across all target outputs is summarized in 4 using NMAE and NRSME; R2 was not used as an evaluation metric for this validation method due its high variance when computed on small validation sets. Overall, our models perform well on melanin predictions, with relatively low NMAEs and NRMSEs. Erythema exhibits higher variance in prediction, likely due to outliers in the dataset that the framework may struggle to generalize to. This is expected as recruiting for diverse erythema levels can be extremely challenging. For CIE LAB values, This disclosure will achieve errors below 2.5 ΔE units, indicating excellent color accuracy that falls within generally accepted thresholds for perceptible color differences. This disclosure will expect higher sampling of minority classes to strengthen these results in future studies. values that can cause large amounts of variance, even within the same measurement site on the same subject.

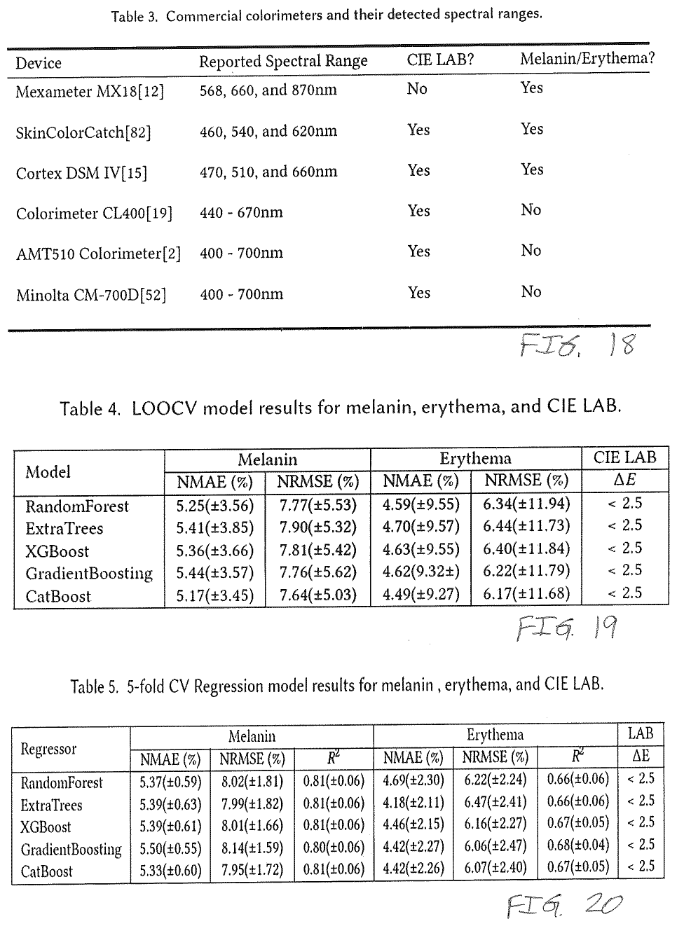

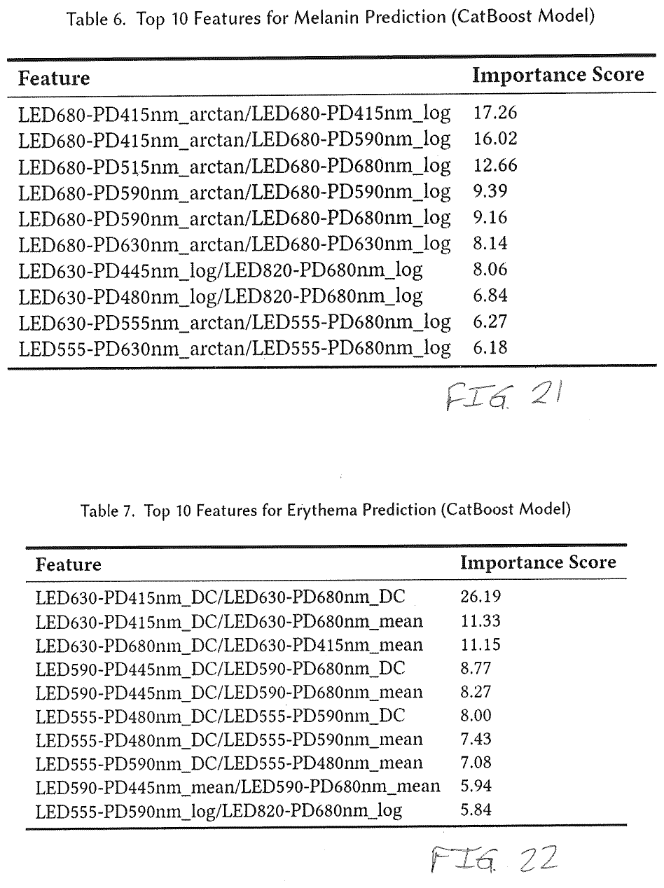

Five (5)-fold CV Results. The results of the five highest performing models using 5-fold cross validation with subject stratification across all target outputs is summarized in Table 5 of FIG. 20. The reported metrics-NMAE, NRMSE, and R2-for each regressor is a mean score +/−standard deviation. The strong and consistent performance across many regressors underscores the strength of the feature-set data in predicting skin constituents. Melanin and erythema achieved less than 6% and 5% NMAE, respectively, relative to the reference measurement across all regression models. For LAB color space evaluation, all models achieved ΔE values below 2.5, demonstrating high color prediction accuracy that meets industry standards for acceptable color differences. Overall, the general accuracy of the framework of this disclosure is relatively strong, especially considering the variance that can occur from measurement to measurement; This disclosure will expect even stronger results with more diverse, targeted recruitment.

Contextualizing Model Accuracy. Although there are no established clinical thresholds for acceptable prediction errors in melanin or erythema, which limits formal clinical interpretation of model performance, the NMAE values achieved by our framework represent small deviations relative to the full physiological ranges of these biomarkers. Melanin index values commonly range from 20 to 120 across skin tones, while erythema indices typically vary from 0 to over 50 depending on inflammation and vascular status. A 5% NMAE corresponds to an error of approximately 1-6 units for melanin (depending on baseline skin tone) and roughly 0.5-2.5 units for erythema. In both cases, these small deviations are unlikely to alter clinical interpretation or influence treatment decisions; Vasudevan et al. [86] reported intra-observer coefficients of variation below 10% across various commercial melanometers and colorimeters, indicating that our measurements fall comfortably within the range of industry-accepted variability.

Accuracy across Skin-Tones and Locations. To evaluate if this accuracy is consistent across skin-tones, this disclosure grouped NMAE and ΔE results based on their ITA classifications; these results are presented in FIG. 6. The analysis demonstrates that our models achieve consistent performance across most skin tones for melanin and erythema predictions, with marginally higher error rates observed in the very light and dark brown categories. These slight variations in accuracy can be attributed to the lower representation of these skin tones in our study population. Very light skin tones likely had higher ΔE values due to their low sample size and the impact of redness that can be seen visibly in these skin-tones. Future studies with more balanced representation across all skin tone categories, particularly in the edge classes, are expected to further improve these results. NMAE and ΔE values across each body location are presented in FIG. 7 below. The errors reveal consistent performance across different body locations, demonstrating the ability of embodiments herein to operate in a location agnostic manner despite the variations in skin-tone distributions seen across body locations. The highest ΔE values were observed in the shoulder and finger measurements. This may be due to the uneven distribution of ITA measurements seen in finger measurements, as well as the scales of these values having much higher ranges, which may cause variation especially in underrepresented groups. This disclosure will expect more stability in these measurements with a larger and more diverse dataset.

Feature Importance. This disclosure evaluated feature importance of the resulting models using their default regression importance metrics. These native techniques revealed that the most important features for melanin and L* prediction were arctan features, specifically in the 600-700 nm range, while the DC features were the most important predictors for erythema, A*, and B*, particularly in the 500-800 nm range. These results coincide with the expected spectral contribution of melanin [46, 64, 93, 94] and erythema [24, 77] found in previous lab studies. A list of top feature importances for each output target and best performing model can be found herein in Tables 6 - 10 of the figures.

Utilizing the PhysioNet Open Oximetry repository, this disclosure will examined differences between measurement accuracy in pulse oximeters relative to arterial blood measurements across skin-tone groups. This disclosure clustered subjects into skin tone groups based on ITA and associated skin-tone classification. Results indicated a significant variation in pulse oximeter measurement accuracy across skin-tones (p-value <0.0001) in both normal and hypoxic conditions across many commercial-grade and hospital-grade devices. MAE is also generally elevated across skin-tones in hypoxic conditions. This finding has been confirmed over a variety of previous works [25, 26, 35, 38, 40, 42, 57, 70, 73, 78, 84]. The model This disclosure will developed as a corrective measure for this error resulted in a significant improvement, achieving an up to 75% reduction in Mean Absolute Error (MAE) across different skin tones, and notably reducing variability among these groups, as illustrated in FIG. 8. Notably, this same MAE decrease was observed in hypoxic conditions, where many racial disparities in pulse oximeter measurements have been reported [25, 26, 35, 38, 40, 42, 57, 70, 73, 78, 84]. This disclosure also confirmed this finding using the complete pulse oximeter dataset, demonstrating that our correction attenuates biases in poor measurements while preserving the accuracy of already reliable readings. Utilizing feature importance techniques, This disclosure discovered the most significant feature in this model was the relation of L* and b* with the calculated pulse oximeter saturation. These results highlight the potential of devices used herein to enhance the accuracy and reduce biases in skin-tones in existing pulse oximetry devices, thereby improving their generalizability across diverse populations.

This disclosure evaluated the pulse oximeter bias corrections across all measurements stratified by skin-tone and found statistically significant differences between groups (p <0.0001), as shown in FIG. 9a. This finding suggests that the model does not apply a uniform correction across all skin tones but instead integrates skin tone information dynamically during prediction. Furthermore, the OpenOximetry dataset contains relatively few dark-skinned fingertips, leading to a distribution that is heavily skewed towards lighter-skinned participants as shown in FIG. 9b. Despite this limitation, the observed trends remain consistent and statistically robust across the full range of skin tone groups, suggesting that incorporating skin tone as a predictive feature in blood oxygen estimation may be key to enabling equitable pulse oximetry.

This disclosures shows future work planned for the framework of this disclosure. This disclosure begins by discussing the overall study limitations and plans to recruit a larger participant pool for algorithm development, with greater representation across skin-tones. Second, this disclosure will explore various applications for the embodiments disclosed herein, with a focus on current pulse oximeters, AI, and wearable devices. Lastly, this disclosure will discuss potential commercialization avenues for the embodiments of this disclosure in the dermatological and cosmetic spaces.

While the embodiments herein were evaluated across a diverse cohort, certain limitations in skin tone representation should be noted. Specifically, participants with very light and dark skin tones were underrepresented in our dataset, particularly in fingertip measurements. This underrepresentation likely contributed to marginally high error rates observed in these groups (FIG. 6), and limits generalizability in edge cases of the ITA scale. While this disclosure employed objective, ITA-based classifications to avoid relying on subjective race labels, the participant pool still skewed toward intermediate skin tones. This skew was partly due to natural population demographics and practical limitations in recruiting individuals at the extremes of the ITA spectrum. This disclosure will believe that future studies, with targeted recruitment efforts, will increase robustness of our framework across racial and ethnic groups.

One of the clear next steps for devices, systems and methods disclosed herein is large-scale validation and qualification. To improve system generalization across various skin-tones and conditions, this disclosure will plan to conduct a clinical trial with recruitment of a larger and more diverse participant base. Targeted recruitment of underrepresented skin-tone groups (such as very light and darker skin-tones) should be conducted to ensure a fair representation across skin-tones. It is important to note that although ITA classifications of individuals help contextualize otherwise abstract values, a diverse set of ITA values within each skin-tone group is equally as vital when recruiting a diverse subject population. Similarly, ITA distributions within each measurement location can also vary quite significantly and should be considered in future studies. Lastly, it would also be of critical importance to validate results using multiple colorimeters and spectrophotometers as ground-truth measures to ensure the device is not biased to a specific colorimeter's results. This disclosure will plan to conduct a validation study with these parameters in mind.

It would also be beneficial for future work to directly recruit for varying erythema levels, as erythema levels can change drastically from participant to participant, as well as over time. Future work should also consider targeted recruitment of individuals with conditions affecting the skin, such melanomas and carcinomas, cyanosis, and PVD; this disclosure could be used in this case to analyze skin composition and monitor changes throughout the progression of these conditions. By integrating melanin and erythema levels associated with various skin and environmental conditions, this disclosure shows valuable insights for the long-term monitoring of various health issues. These large-scale validation studies can also enable the examination of UV exposure's effects on melanin and erythema levels. It would be particularly valuable to observe relative changes between melanin and erythema, especially in dermatological or environmental conditions where these values can change over time. While localized UV exposure effects are well-documented, [7, 72], the systemic impacts of UV radiation on overall skin health and its relationship to melanin and erythema changes across different body sites remain largely unexplored. Furthermore, the effects of UV exposure on other biomarkers, such as heart rate and oxygenation, can be examined more easily using the framework disclosed herein.

Optical Sensor Considerations. Environmental factors such as ambient light variations and participant movement can significantly impact optical measurements in wearable devices [9]. While these conditions have been thoroughly explored within processing strategies that show promise in maintaining measurement integrity in real-world conditions. First, our baseline DC filtering approach effectively removes low-frequency noise from ambient light variations and gradual posture changes. The normalization features this disclosure developed, particularly the optical density ratios between different sensor pairs, further reduces the impact of motion artifacts by considering relative rather than absolute changes. Additionally, the multi-wavelength nature of our system provides inherent advantages for noise reduction. By capturing spectral data across multiple wavelengths simultaneously, our system achieves a form of measurement redundancy. This multi-modal approach potentially increases signal fidelity and robustness to environmental noise, as artifacts typically don't affect all wavelengths equally. However, this disclosure will note that the specific contributions of multi-wavelength measurements to noise reduction, particularly in different lighting conditions and movement scenarios, warrants further investigation. Future work should systematically evaluate these effects through controlled studies comparing single versus multi-wavelength performance under various environmental conditions.

While the OpenOximetry database is a valuable resource for evaluating pulse oximeter performance across different skin tones, it has certain limitations. One key constraint is that PPG waveform data is not available for all encounters, and even when it is present, it is not time-synchronized with arterial blood gas measurements. This lack of synchronization reduces the ability to directly analyze pulse oximetry signal dynamics in relation to skin tone. Additionally, the dataset is skewed towards lighter-skinned individuals in fingertip measurements, limiting its representativeness for darker skin tones. This disclosure observed a similar trend in our study, despite targeted recruitment of individuals with darker skin tones. Many fingertip measurements still fell within the intermediate to tan range, making it challenging to obtain sufficient data from participants with dark-skinned fingertips. This recruitment difficulty should be considered in future studies to ensure a more balanced dataset that better represents the full spectrum of skin tones.

Due to limitations in acquiring the blood oxygen algorithms for pulse oximeters as well as raw PPG waveforms in the OpenOximetry database, a regression model was built on top of the existing saturation values, a methodology of this disclosure will believe to be suboptimal. Ideally, the skin-tone correction would occur during the SpO2 calculation at the PPG signal level, where a baseline measurement would be taken to assess skin-tone at the site of data collection and used directly while calculating the current saturation level. This would be analogous to current PPG LED controllers, where the LED intensity is modulated based on the surrounding light measured by the device's PDs. This method is typically used to increase light intensity when the skin-tone is dark, which can be aided by also taking the skin-tone measurements. However, recent studies have suggested that modulating LED intensity can distort pulse oximetry measurements, especially in hypoxic conditions [88]. As such, a multi-spectrum approach, such as the one used in the embodiments herein may be preferred, especially considering the reduction in observed MAE through the use of our model. To integrate this correction approach into existing pulse oximeter devices, manufacturers could extend the spectral range beyond red and infrared to include wavelengths relevant to melanin absorption and utilize a prediction algorithm for skin-tone constituents, specifically CIE LAB values.

As a standalone wearable device, devices disclosed herein can be tailored to skin analysis by incorporating sensors with LED and PD wavelengths that provide a greater spectral resolution in the 500-900 nm range. This may aid in improving the accuracy of our algorithm, as melanin and erythema have high absorption bands in this area, thus allowing us to effectively differentiate between the two more effectively. This may also help in analyzing micro-trends in skin, which could be crucial for certain dermatological conditions. Despite this limitation, our algorithm has shown great results using a commercial solution, which can be improved upon with this modification. Device sensors and algorithms presented in this disclosure can be readily adapted to other wearable wrist-worn devices that utilize optical spectroscopy, such as the Apple Watch [3] or Google Pixel Watch [34]. Both devices have recently included multi-spectrum sensors in these devices to improve optical biomarker detection. This disclosure can be utilized in a similar fashion by expanding the sensor resolution to include wavelengths pertinent to skin composition measurement, along with the model of the embodiments herein. This would aid in detection of optically obtained biomarkers across skin-tones, while also enabling skin monitoring continuously on the wrist. This disclosure will recognize that UV monitoring on the wrist may be counter-intuitive, especially considering the site of analysis would be covered by the watch, and thus potential UV overexposure may not be identified. However, it may be helpful to analyze melanin and erythema levels in this case, as there may be compositional changes in the skin due to this exposure that are not necessarily evident visually. Melanocytes—cells that produce melanin—have been shown to release signaling molecules that influence each other's functions. Upon UV exposure, melanocytes can modulate the behavior of surrounding cells, contributing to an integrated defense mechanism against UV-damage [11].

Embodiments herein can allow further exploration into the effects of surrounding UV radiation in this context, which has remained largely unexplored.

Embodiments of this disclosure have several potential dermatological use cases that This disclosure will have identified. The first of these is a wearable patch that can be prescribed by a physician for either short or long-term monitoring of a potential skin disorder. For example, during a telehealth visit, a dermatologist might examine a patient's skin; if further monitoring is required, the physician could prescribe embodiments of this disclosure to examine the affected area and monitor it remotely, thereby preventing a potentially expensive visit and/or invasive biopsy, reducing costs to both the patient and the healthcare system. These patches could also be used to monitor allergic reactions or infections at incision sites, offering significant applications in the clinic, such as for peri-operative monitoring, or during ENT appointments where potential allergies are being determined. Typically, allergies are determined via skin or blood tests, where the skin is exposed to a suspected allergen. Allergies are then determined by examining the width and overall extent of resulting erythema, which is analyzed and recorded by the attending physician. This is a subjective measure, and it may be more accurately quantified by the devices, systems and methods disclosed herein. Furthermore, since the embodiments of this disclosure can measure the skin continuously, the progression of erythema could be tracked over time. This application could also be extended towards monitoring of cosmetic laser treatments, where colorimeters are often used to determine the type of treatment needed. The continuous nature of data collection as disclosed herein can provide crucial insights to enhance these therapies to maximize therapeutic benefit and patient comfort. Systems, methods and devices of the embodiments herein have potential cosmetic applications for clinical monitoring and substantiating the efficacy of cosmetic products. Cosmetic companies often perform small to large-scale clinical trials where products are tested on a subject population to examine skin traits such as overall brightening for products like serums and creams.

However, a limiting factor in these analyses is many of these trials are conducted on-site using colorimeters, which limits the ability to analyze skin reactions long-term. Often, this is solved by monitoring patients in-clinic over time, which increases subject burden and overall cost. This disclosure could be used in a small wearable patch form-factor for this application, enabling analysis of the skin in unapplied and applied product areas to enhance research on product efficacy and overall claims substantiation. Since the data collected is continuous, scientific comparison would be more robust, and these comparisons can even be conducted remotely. This capability would greatly simplify data collection efforts, provide potentially enhanced data on skin reaction to various products, and reduce the need for prolonged in-clinic stays.

Current methods that quantify melanin, erythema, and/or skin-tone often suffer from being highly subjective, expensive, non-continuous or impractical for daily use. The potential for a non-invasive, wearable, and continuous quantitative skin measurement device has implications for skin monitoring, as well as correcting for racial biases in pulse oximeter measurements. This disclosure introduced a novel wearable framework that utilizes optical spectroscopy to quantify melanin, erythema, and skin-tone. The results demonstrate that the systems, methods and devices as shown herein can accurately quantify melanin, erythema, and CIE L*, a*, and b* values, achieving a normalized mean absolute error (NMAE) of 5.33% for melanin and 4.18% for erythema, with ΔE values below 2.5 when compared to a state-of-the-art commercial colorimeter. Furthermore, this disclosure developed an algorithm leveraging outputs to correct pulse oximeter inaccuracies associated with skin tone, leading to up to a 75% reduction in MAE for hypoxic readings. These findings underscore the potential that this disclosure brings as both a continuous skin monitoring solution and a corrective system for improving the accuracy of wearable medical devices that rely on skin perfusion measurements, particularly in addressing disparities in pulse oximetry across diverse skin tones.

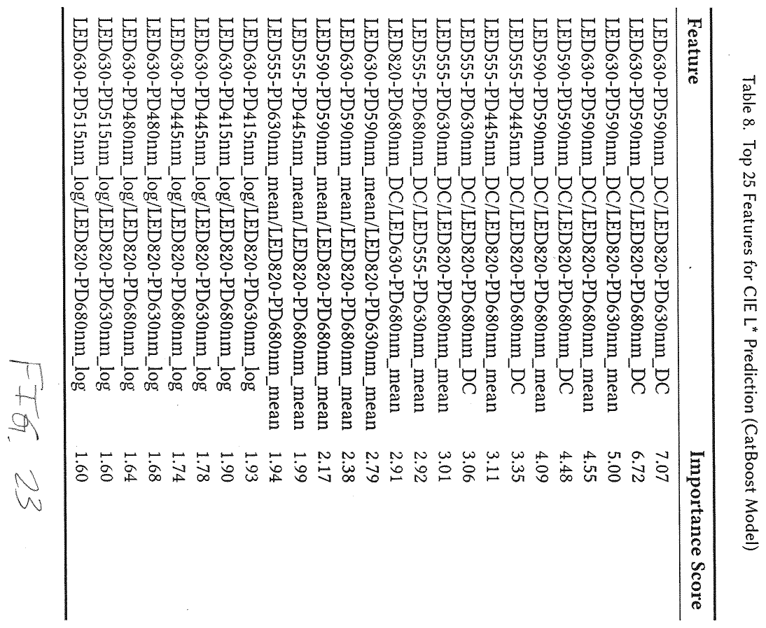

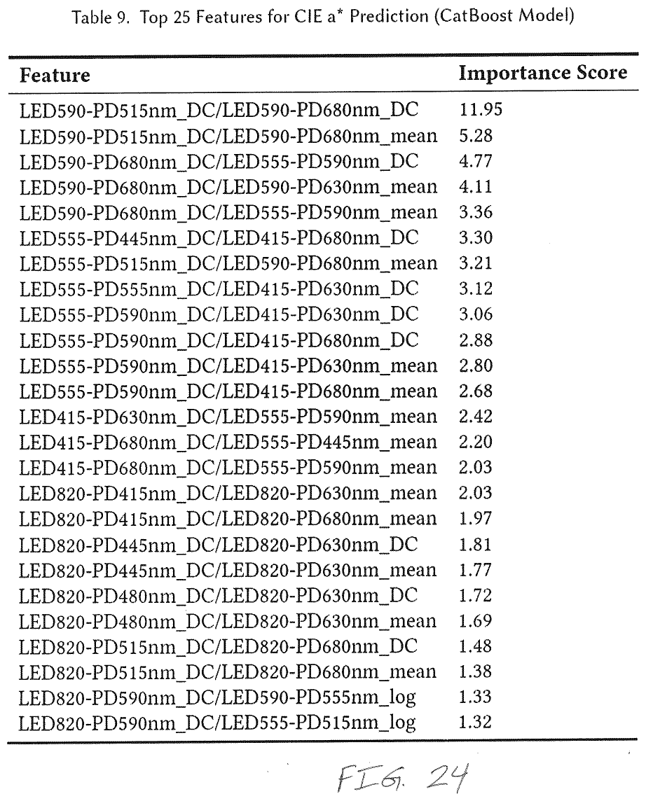

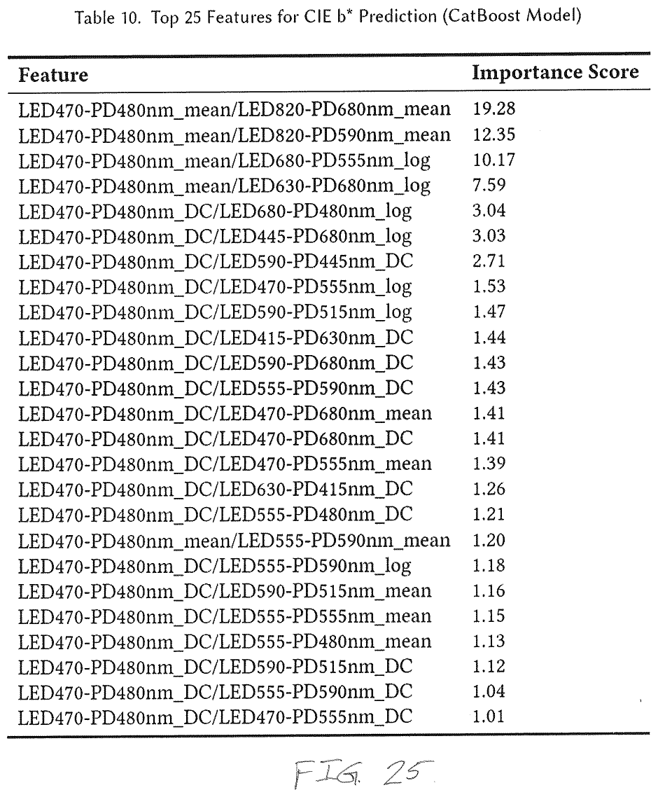

The following tables summarize the top 10 features selected for each output and their relative importance scores from the final CatBoost regression model. Feature names are abbreviated for clarity (e.g., LED680-PD415 nm_arctan refers to the arctan of the 415 nm PD response when the 680 nm LED is illuminated.). See Tables 6-10 at FIGS. 21-25.

In one embodiment, a system for collecting skin tone data for a subject includes a plurality of light emitting diodes (LEDs) 105, 115 directing a range of light wavelengths onto skin. A corresponding set of photodiodes (PDs) 106, 116 capture spectral data for a corresponding range of wavelengths from reflected light from the skin. A computer having a processor connected to computer memory stores software 107, 117 that when executed performs a computer implemented method with steps of extracting features 108, 118 from the spectral data with a signal processing component of the software and applying the features from the spectral data to a machine learning model 109 that identifies physiology data 110 and color tone data 112 for the skin.

In another embodiment, the photodiodes capture the spectral data on a continuous basis.

In another embodiment, extracting features includes calculating mean values 119 of each photodiode response over a measurement period.

In another embodiment, extracting features includes calculating direct current (DC) content 20 of each PD response over a measurement period.

In another embodiment, extracting features includes calculating an individual typology angle (ITA) 122 for the skin.

In another embodiment, extracting features includes calculating ratios 121 of combinations of LED outputs and photodiode responses.

In another embodiment, combinations of LED outputs and photodiode responses different wavelengths of light.

In another embodiment, the range of wavelengths of the LEDs is within the visible spectrum.

In another embodiment, the spectral data is three-dimensional spectral data.

In another embodiment, the three-dimensional spectral data includes an L* axis, and a* axis, and a b* axis.

In another embodiment, extracting features includes calculating an individual typology angle (ITA) for the skin with L* and b* components from the three-dimensional spectral data.

In another embodiment, the individual typology angle (ITA) is used to classify the color tone of the skin according to an ITA range.

In another embodiment, the machine learning model identifies the physiology data of the skin with a regression algorithm.

In another embodiment, the regression algorithm assigns a quantitative value to either or both of melanin level or erythema level of the skin.

In another embodiment, the system further comprising another piece of hardware or a medical device such as pulse oximeter that is calibrated 113 according to at least one of a skin color tone, a melanin level, or an erythema level.

In some aspects, the disclosed technology relates to systems, methods, and computer-readable medium improving skin tone analysis and using that data in other machinses. Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

This disclosure represents a significant advancement in skin assessment technology, utilizing state-of-the-art machine learning techniques to optimize related hardware. This disclosure integrates a sophisticated neural network-based agent that performs complex analysis of multi-modal time series data from sources

It should be appreciated that any element, part, section, subsection, or component described with reference to any specific embodiment above may be incorporated with, integrated into, or otherwise adapted for use with any other embodiment described herein unless specifically noted otherwise or if it should render the embodiment device non-functional. Likewise, any step described with reference to a particular method or process may be integrated, incorporated, or otherwise combined with other methods or processes described herein unless specifically stated otherwise or if it should render the embodiment method nonfunctional. Furthermore, multiple embodiment devices or embodiment methods may be combined, incorporated, or otherwise integrated into one another to construct or develop further embodiments of the disclosure described herein.

It should be appreciated that any of the components or modules referred to with regards to any of the present disclosure embodiments discussed herein, may be integrally or separately formed with one another. Further, redundant functions or structures of the components or modules may be implemented. Moreover, the various components may be communicated locally and/or remotely with any user/clinician/patient or machine/system/computer/processor. Moreover, the various components may be in communication via wireless and/or hardwire or other desirable and available communication means, systems and hardware. Moreover, various components and modules may be substituted with other modules or components that provide similar functions.

It should be appreciated that the device and related components discussed herein may take on all shapes along the entire continual geometric spectrum of manipulation of x, y and z planes to provide and meet the anatomical, environmental, and structural demands and operational requirements. Moreover, locations and alignments of the various components may vary as desired or required.

It should be appreciated that various sizes, dimensions, contours, rigidity, shapes, flexibility and materials of any of the components or portions of components in the various embodiments discussed throughout may be varied and utilized as desired or required.

It should be appreciated that while some dimensions are provided on the aforementioned figures, the device may constitute various sizes, dimensions, contours, rigidity, shapes, flexibility and materials as it pertains to the components or portions of components of the device, and therefore may be varied and utilized as desired or required.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, or method steps, even if the other such compounds, material, particles, or method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. All references cited and discussed in this specification are incorporated herein by reference in their entireties and to the same extent as if each reference was individually incorporated by reference.

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g. rat, dog, pig, monkey), etc. It should be appreciated that the subject may be any applicable human patient, for example.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

Additional descriptions of aspects of the present disclosure will now be provided with reference to the accompanying drawings. The drawings form a part hereof and show, by way of illustration, specific embodiments or examples.

### REFERENCES

[1] R. Abdlaty, J. Hayward, T. Farrell, and Q. Fang. 2021. Skin erythema and pigmentation: a review of optical assessment techniques. Photodiagnosis Photodyn Ther 33 (2021), 102127. doi:10.1016/j.pdpdt.2020.102127 Epub 2020 Dec. 1.
[2] Amtast. [n. d.]. AMT 510 Colorimeter. Colorimeter used for scientific and industrial color measurement. https://amtast.com/content-93.html
[3] Apple. 2024. Apple Watch. https://www.apple.com/watch/
[4] M Baquié and B Kasraee. 2014. Discrimination between cutaneous pigmentation and erythema: comparison of the skin colorimeters Dermacatch and Mexameter. Skin Res. Technol. 20, 2 (May 2014), 218-227.
[5] B Bent, B A Goldstein, W A Kibbe, and J P Dunn. 2020. Investigating sources of inaccuracy in wearable optical heart rate sensors. NPJ Digital Medicine 3 (February 2020), 18. doi:10.1038/s41746-020-0226-6
[6] D Z Bliss, O Gurvich, K Savik, et al. 2017. Racial and ethnic disparities in the healing of pressure ulcers present at nursing home admission. Archives of Gerontology and Geriatrics 72 (2017), 187-194.
[7] Michaela Brenner and Vincent J Hearing. 2008. The protective role of melanin against UV damage in human skin. Photochemistry and Photobiology 84, 3 (May-June 2008), 539-549. doi:10.1111/j.1751-1097.2007.00226.x
[8] A M Cabanas, M Fuentes-Guajardo, K Latorre, D León, and P Martín-Escudero. 2022. Skin Pigmentation Influence on Pulse Oximetry Accuracy: A Systematic Review and Bibliometric Analysis. Sensors 22, 9 (April 2022), 3402. doi:10.3390/s22093402
[9] D. Castaneda, A. Esparza, M. Ghamari, C. Soltanpur, and H. Nazeran. 2018. A review on wearable photoplethysmography sensors and their potential future applications in health care. International Journal of Biosensors & Bioelectronics 4, 4 (2018), 195-202. doi:10.15406/ijbsbe.2018.04.00125 Epub 2018 Aug. 6.
[10] E D Chan, M M Chan, and M M Chan. 2013. Pulse oximetry: understanding its basic principles facilitates appreciation of its limitations. Respiratory Medicine 107, 6 (June 2013), 789-799. doi:10.1016/j. rmed.2013.02.004 Epub 2013 Mar. 13.
[11] Mirosława Cichorek, Małgorzata Wachulska, Agnieszka Stasiewicz, and Agnieszka Tyminska. 2013. Skin melanocytes: biology and development. Postepy Dermatologii i Alergologii 30, 1 (2013), 30-41. doi:10.5114/pdia.2013.33376
[12] CKElectronic. [n. d.]. Mexameter MX18. Device used for measuring skin melanin and erythema. https://www.courage-khazaka.//en/scientific-products/occupational-health/occupational-health/169-mexameter-e
[13] P J Colvonen. 2021. Response To: Investigating sources of inaccuracy in wearable optical heart rate sensors. NPJ Digital Medicine 4, 1 (February 2021), 38. doi:10.1038/s41746-021-00408-5
[14] P J Colvonen, P N DeYoung, N A Bosompra, and R L Owens. 2020. Limiting racial disparities and bias for wearable devices in health science research. Sleep 43, 10 (October 2020), zsaa159. doi:10.1093/sleep/zsaa159
[15] Cortex. 2024. Cortex DSM 4. Digital spectrophotometer for dermatological assessments. https://www.cortex.dk/skin-analysis/colorimeter-dsm-4/
[16] S Del Bino and F Bernerd. 2013. Variations in skin colour and the biological consequences of ultraviolet radiation exposure. The British Journal of Dermatology 169, Suppl 3 (2013), 33-40. doi:10.1111/bjd.12529
[17] DermLite 2024. DermLite DL5. DermLite. https://dermlite.com/products/dermlite-dl5
[18] L. Dhoonmoon and J. Fletcher. 2022. Assessing skin tones in practice: results of an international survey. Wounds International 13, 2 (2022), 6-9.
[19] CK Electronic. 2024. COLORIMETERR CL 400. Precision colorimeter for color consistency and quality control. https://www.enviroderm.co.uk/products/colorimeter-cl-400
[20] A. Elyas and M. Bradley. 2021. Viktigt att beakta att hudåkommor kan variera beroende på hudtyp [An introduction to skin disease in melanin rich skin]. Lakartidningen 118 (2021), 20208. Swedish.
[21] J S C English, R S Dawe, and J Ferguson. 2003. Environmental effects and skin disease. British Medical Bulletin 68 (2003), 129-142. doi:10.1093/bmb/ldg026
[22] European Pressure Ulcer Advisory Panel and National Pressure Ulcer Advisory Panel and Pan-Pacific Pressure Injury Alliance Year of Publication. Prevention and Treatment of Pressure Ulcers/Injuries: Quick Reference Guide. European Pressure Ulcer Advisory Panel and National Pressure Ulcer Advisory Panel and Pan-Pacific Pressure Injury Alliance. URLifavailable Accessed: insert access date here.
[23] Thomas Fahrni, Michael Kuhn, Philipp Sommer, Roger Wattenhofer, and Samuel Welten. 2011. Sundroid: solar radiation awareness with Smartphones. In Proceedings of the 13th International Conference on Ubiquitous Computing (Beijing, China) (UbiComp ‘11). Association for Computing Machinery, New York, NY, USA, 365-374. doi:10.1145/2030112.2030162
[24] D Fajuyigbe, A Coleman, R P E Sarkany, A R Young, and A W Schmalwieser. 2018. Diffuse Reflectance Spectroscopy as a Reliable Means of Comparing Ultraviolet Radiation-induced Erythema in Extreme Skin Colors. Photochemistry and Photobiology 94, 5 (September 2018), 1066-1070. doi:10.1111/php.12947 Epub 2018 Jul. 4.
[25] A Fawzy, T D Wu, K Wang, M L Robinson, J Farha, A Bradke, S H Golden, Y Xu, and B T Garibaldi. 2022. Racial and Ethnic Discrepancy in Pulse Oximetry and Delayed Identification of Treatment Eligibility Among Patients With COVID-19. JAMA Internal Medicine 182, 7 (2022), 730-738. doi:10.1001/jamainternmed.2022.1906 Erratum in: JAMA Intern Med. 2022 October 1;182(10):1108.
[26] A Fawzy, T D Wu, K Wang, K E Sands, A M Fisher, S A Arnold Egloff, J D DellaVolpe, T J Iwashyna, Y Xu, and B T Garibaldi. 2023. Clinical Outcomes Associated With Overestimation of Oxygen Saturation by Pulse Oximetry in Patients Hospitalized With COVID-19. JAMA Network Open 6, 8 (2023), e2330856. doi:10.1001/jamanetworkopen.2023.30856
[27] J. R. Feiner, J. W. Severinghaus, and P. E. Bickler. 2007. Dark skin decreases the accuracy of pulse oximeters at low oxygen saturation: the effects of oximeter probe type and gender. Anesth Analg 105, 6 Suppl (2007), S18-S23. doi:10.1213/01.ane.0000285988.35174.d9
[28] T. B. Fitzpatrick. 1975. Soleil et peau. Journal of Medicine Esthetique and Dermatological Surgery 2 (1975), 33-34. Sun and skin.
[29] A D et al. Fix. 2000. Racial differences in reported Lyme disease incidence. American journal of epidemiology 152, 8 (2000), 756-9. doi:10.1093/aje/152.8.756
[30] Nicholas Fong et al. 2024. OpenOximetry Repository. PhysioNet. doi:10.13026/cc78-ad74
[31] Food and Drug Administration. 2025. Pulse Oximeters for Medical Purposes-Non-Clinical and Clinical Performance Testing, Labeling, and Premarket Submission Recommendations: Draft Guidance for Industry and Food and Drug Administration Staff. Draft Guidance. U.S. Department of Health and Human Services. Document issued on Jan. 7, 2025.
[32] K F Francis. 2023. Assessment and Identification of skin disorders in skin of color: an integrative review. Journal of Wound Ostomy and Continence Nursing 50, 2 (2023), 107-114.
[33] A. Goldberger, L. Amaral, L. Glass, J. Hausdorff, P. C. Ivanov, R. Mark, J. E. Mietus, G. B. Moody, C. K. Peng, and H. E. Stanley. 2000. PhysioBank, PhysioToolkit, and PhysioNet: Components of a new research resource for complex physiologic signals. Circulation 101, 23 (2000), e215-e220.
[34] Google. 2024. Google Pixel Watch. https://store.google.com/us/product/pixel_watch_2?hl=en-US
[35] E R Gottlieb, J Ziegler, K Morley, B Rush, and L A Celi. 2022. Assessment of Racial and Ethnic Differences in Oxygen Supplementation Among Patients in the Intensive Care Unit. JAMA Internal Medicine 182, 8 (Aug 2022), 849-858. doi:10.1001/jamainternmed.2022.2587
[36] M. K. Gudelunas, M. Lipnick, C. Hendrickson, S. Vanderburg, B. Okunlola, I. Auchus, J. R. Feiner, and P. E. Bickler. 2024. Low Perfusion and Missed Diagnosis of Hypoxemia by Pulse Oximetry in Darkly Pigmented Skin: A Prospective Study. Anesth Analg 138, 3 (2024), 552-561. doi:10.1213/ANE.0000000000006755 Epub 2023 Dec. 18.
[37] Tarek Hamid, Insup Lee, and Amanda Watson. 2023. SpectraVue-An Interactive Web Application Enabling Rapid Data Visualization and Analysis for Wearable Spectroscopy Research (UbiComp/ISWC ‘23 Adjunct). Association for Computing Machinery, New York, NY, USA, 146-150. doi:10.1145/3594739.3610709
[38] N R Henry, A C Hanson, P J Schulte, N S Warner, M N Manento, T J Weister, and M A Warner. 2022. Disparities in Hypoxemia Detection by Pulse Oximetry Across Self-Identified Racial Groups and Associations With Clinical Outcomes. Critical Care Medicine 50, 2 (February 2022), 204-211. doi:10.1097/CCM.0000000000005394
[39] L C Hsu, S Hsu, T H Tan, C H Cheng, and C C Chang. 2022. Developing Low-Cost Mobile Device and Apps for Accurate Skin Spectrum Measurement via Low-Cost Spectrum Sensors and Deep Neural Network Technology. Sensors 22, 22 (November 2022), 8844. doi:10.3390/s22228844
[40] H Jamali, L T Castillo, C C Morgan, J Coult, J L Muhammad, O O Osobamiro, E C Parsons, and R Adamson. 2022. Racial Disparity in Oxygen Saturation Measurements by Pulse Oximetry: Evidence and Implications. Annals of the American Thoracic Society 19, 12 (December 2022), 1951-1964. doi:10.1513/AnnalsATS.202203-270CME
[41] V G Kanellis. 2019. A review of melanin sensor devices. Biophysical Reviews 11, 6 (December 2019), 843-849. doi:10.1007/s12551-019-00581-8
[42] S W Ketcham, M C Konerman, V D Marshall, and S K Adie. 2024. Racial Bias in Pulse Oximetry Measurement: Considerations in Patients With Heart Failure. Circulation: Cardiovascular Quality and Outcomes 17, 3 (March 2024), e010390. doi:10.1161/CIRCOUTCOMES.123.010390
[43] A. K. Khanna, J. Beard, S. Lamminmäki, J. Närväinen, N. Antaki, and H. O. Yapici. 2024. Assessment of skin pigmentation-related bias in pulse oximetry readings among adults. J Clin Monit Comput 38, 1 (2024), 113-120. doi:10.1007/s10877-023-01095-1 Epub 2023 Oct. 26.
[44] D Koerber, S Khan, T Shamsheri, A Kirubarajan, and S Mehta. 2023. Accuracy of Heart Rate Measurement with Wrist-Worn Wearable Devices in Various Skin Tones: a Systematic Review. Journal of Racial and Ethnic Health Disparities 10, 6 (December 2023), 2676-2684. doi:10.1007/s40615-022-01446-9 Epub 2022 Nov. 14.
[45] N. Kollias and A. Baqer. 1985. Spectroscopic characteristics of human melanin in vivo. Journal of Investigative Dermatology 85, 1 (1985), 38-42. doi:10.1111/1523-1747.ep12275011
[46] N Kollias and A H Baqer. 1987. Absorption mechanisms of human melanin in the visible, 400-720 nm. Journal of Investigative Dermatology 89, 4 (October 1987), 384-388. doi:10.1111/1523-1747.ep12471764
[47] La Roche-Posay 2024. My UV Patch. La Roche-Posay by L'Oréal. https://www.lorealtechincubator.com/myuvpatch A stretchable skin sensor that monitors UV radiation exposure and works with a mobile app.
[48] M Langeveld, L S van de Lande, E O'Sullivan, B van der Lei, and J A van Dongen. 2022. Skin measurement devices to assess skin quality: A systematic review on reliability and validity. Skin Research and Technology 28, 2 (March 2022), 212-224. doi: 10.1111/srt.13113
[49] LogicInk 2024. LogicInk. LogicInk. https://logicink.com/Creates wearable sensors that provide visual indications of health and environmental exposures, including UV exposure.
[50] S Maya-Enero, J Candel-Pau, J Garcia-Garcia, A M Gimenez-Arnau, and M A Lopez-Vilchez. 2020. Validation of a neonatal skin color scale. European Journal of Pediatrics 179, 9 (2020), 1403-1411.
[51] H E McCreath, B M Bates-Jensen, G Nakagami, et al. 2016. Use of Munsell Color Charts to measure skin tone objectively in nursing home residents at risk for pressure ulcer development. Journal of Advanced Nursing 72, 9 (2016), 2077-2085.
[52] Konica Minolta. [n. d.]. Konica Minolta CM-700d Spectrophotometer. Spectrophotometer for accurate color measurement in a wide range of industries. https://sensing.konicaminolta.//us/products/cm-700d-spectrophotometer/
[53] MoleScope 2024. MoleScope. MetaOptima Technology Inc. https://www.molescope./m/
[54] Ellis Monk. 2023. The Monk Skin Tone Scale. https://osf.//preprints/socarxiv/Web.
[55] Sarah Mosca and Aldo Morrone. 2023. Human Skin Pigmentation: From a Biological Feature to a Social Determinant. Healthcare 11, 14 (2023). doi:10.3390/healthcare11142091
[56] M Nitzan, A Romem, and R Koppel. 2014. Pulse oximetry: fundamentals and technology update. Medical Devices (Auckland, NZ) 7 (8 Jul. 2014), 231-239. doi:10.2147/MDER. S47319
[57] O E Okunlola, M S Lipnick, P B Batchelder, M Bernstein, J R Feiner, and P E Bickler. 2022. Pulse Oximeter Performance, Racial Inequity, and the Work Ahead. Respiratory Care 67, 2 (February 2022), 252-257. doi:10.4187/respcare.09795
[58] R. L. Olson, J. Gaylor, and M. A. Everett. 1973. Skin color, melanin, and erythema. Arch Dermatol 108, 4 (1973), 541-544.
[59] J. P. Ortonne. 2002. Photoprotective properties of skin melanin. Br. J. Dermatol. 146 (2002), 7-10. doi: 10.1046/j.1365-2133.146.s61.3.x
[60] Pantone 2024. Pantone SkinTone Guide. Pantone. https://www.pantone.com/products/fashion-home-interiors/pantone-skintone-guide
[61] E M Polfer, R M Zimmerman, E Tefera, R D Katz, J P Higgins, and Jr. Means, K R. 2018. The effect of skin pigmentation on determination of limb ischemia. Journal of Hand Surgery (American Volume) 43, 1 (2018), 24-32.
[62] M. L. Ramsey and S. Rostami. 2023. Skin Biopsy. StatPearls [Internet] (2023). https://www.ncbi.nlm.nih.gov/books/NBK470464/Treasure Island (FL): StatPearls Publishing; 2024 Jan.
[63] P A Riley. 1997. Melanin. International Journal of Biochemistry & Cell Biology 29, 11 (November 1997), 1235-1239. doi:10.1016/s1357-2725(97)00013-7
[64] I Sadiq, N Kollias, and A Baqer. 2019. Spectroscopic observations on human pigmentation. Photodermatology, Photoimmunology & Photomedicine 35, 6 (November 2019), 415-419. doi:10.1111/phpp.12474 Epub 2019 May 24.
[65] L K Saladin and J S Krause. 2009. Pressure ulcer prevalence and barriers to treatment after spinal cord injury: comparisons of four groups based on race-ethnicity. Neurorehabilitation 24, 1 (2009), 57-66.
[66] C J Santiago, V W Weedn, and F J Diaz. 2022. The impact of skin color on the recognition of blunt force injuries. American Journal of Forensic Medicine and Pathology 43, 3 (2022), 220-224.
[67] K N Scafide, M C Narayan, and L Arundel. 2020. Bedside technologies to enhance the early detection of pressure injuries: a systematic review. Journal of Wound Ostomy and Continence Nursing 47, 2 (2020), 128-136.
[68] D. I. Schlessinger, M. D. Anoruo, and J. Schlessinger. 2023. Biochemistry, Melanin. StatPearls [Internet] (2023). Updated 2023 May 1; Treasure Island (FL): StatPearls Publishing; 2024 January. Available from: https://www.ncbi.nlm.nih.gov/books/NBK459156.
[69] K Setchfield, A Gorman, A H R W Simpson, M G Somekh, and A J Wright. 2024. Effect of skin color on optical properties and the implications for medical optical technologies: a review. Journal of Biomedical Optics 29, 1 (January 2024), 010901. doi:10.1117/1.JBO.29.1.010901 Epub 2024 Jan. 24.
[70] M Sharma, A W Brown, N M Powell, N Rajaram, L Tong, P M Mourani, and M Schootman. 2024. Racial and skin color mediated disparities in pulse oximetry in infants and young children. Paediatric Respiratory Reviews (5 Jan. 2024). doi:10.1016/j. prrv.2023.12.006 Epub ahead of print.
[71] A. Shcherbina, C. M. Mattsson, D. Waggott, H. Salisbury, J. W. Christle, T. Hastie, M. T. Wheeler, and E. A. Ashley. 2017. Accuracy in Wrist-Worn, Sensor-Based Measurements of Heart Rate and Energy Expenditure in a Diverse Cohort. J Pers Med 7, 2 (2017), 3. doi:10.3390/jpm7020003
[72] Seiichiro Shono, Genji Imokawa, Yoshihiro Yada, and Mitsuru Kimura. 1985. The relationship of skin color, UVB-induced erythema, and melanogenesis. The Journal of Investigative Dermatology 84, 4 (1985), 265-267. doi: 10.1111/1523-1747.ep12265342
[73] M W Sjoding, R P Dickson, T J Iwashyna, S E Gay, and T S Valley. 2020. Racial Bias in Pulse Oximetry Measurement. New England Journal of Medicine 383, 25 (December 2020), 2477-2478. doi:10.1056/NEJMc2029240 Erratum in: N Engl J Med. 2021 Dec. 23; 385(26): 2496.
[74] SkinVision 2024. SkinVision. SkinVision. https://www.skinvision.com/
[75] M S Sommers, Y Regueira, D A Tiller, et al. 2019. Understanding rates of genital-anal injury: role of skin color and skin biomechanics. Journal of Forensic and Legal Medicine 66 (2019), 120-128.
[76] S E Sonenblum, R Patel, S Phrasavath, S Xu, and B M Bates-Jensen. 2023. Using Technology to Detect Erythema Across Skin Tones. Advances in Skin & Wound Care 36, 10 (October 2023), 524-533. doi: 10.1097/ASW.0000000000000043
[77] G N Stamatas, B Z Zmudzka, N Kollias, and J Z Beer. 2008. In vivo measurement of skin erythema and pigmentation: new means of implementation of diffuse reflectance spectroscopy with a commercial instrument. British Journal of Dermatology 159, 3 (September 2008), 683-690. doi:10.1111/j.1365-2133.2008.08642.x Epub 2008 May 28.
[78] S E K Sudat, P Wesson, K F Rhoads, et al. 2023. Racial disparities in pulse oximeter device inaccuracy and estimated clinical impact on COVID-19 treatment course. American Journal of Epidemiology 192, 5 (2023), 703-713.
[79] S. E. K. Sudat, P. Wesson, K. F. Rhoads, S. Brown, N. Aboelata, A. R. Pressman, A. Mani, and K. M. J. Azar. 2023. Racial Disparities in Pulse Oximeter Device Inaccuracy and Estimated Clinical Impact on COVID-19 Treatment Course. Am J Epidemiol 192, 5 (2023), 703-713. doi:10.1093/aje/kwac164
[80] Sun Index 2024. Sun Index Wearable Watch. Sun Index. https://sunindex.co/A wearable device that provides real-time UV index monitoring to help manage sun exposure..
[81] Jerry Tan, Hong Liu, James J. Leyden, and Matthew J. Leoni. 2014. Reliability of Clinician Erythema Assessment grading scale. Journal of the American Academy of Dermatology 71, 4 (2014), 760-763. doi:10.1016/j. jaad.2014.05.033
[82] Delfin Technologies. 2024. Delfin SkinColorCatch. Portable device for skin color measurement. https://delfintech.com/products/skincolorcatch/
[83] K Tekin, M Karadogan, S Gunaydin, and K Kismet. 2023. Everything About Pulse Oximetry-Part 1: History, Principles, Advantages, Limitations, Inaccuracies, Cost Analysis, the Level of Knowledge About Pulse Oximeter Among Clinicians, and Pulse Oximetry Versus Tissue Oximetry. Journal of Intensive Care Medicine 38, 9 (September 2023), 775-784. doi:10.1177/08850666231185752 Epub 2023 Jul. 12.
[84] V S M Valbuena, R P Barbaro, D Claar, T S Valley, R P Dickson, S E Gay, M W Sjoding, and T J Iwashyna. 2022. Racial Bias in Pulse Oximetry Measurement Among Patients About to Undergo Extracorporeal Membrane Oxygenation in 2019-2020: A Retrospective Cohort Study. Chest 161, 4 (April 2022), 971-978. doi: 10.1016/j. chest.2021.09.025
[85] Martijn van der Wal, Monica Bloemen, Pauline Verhaegen, Wim Tuinebreijer, Henrica de Vet, Paul van Zuijlen, and Esther Middelkoop. 2013. Objective color measurements: clinimetric performance of three devices on normal skin and scar tissue. J. Burn Care Res. 34, 3 (May 2013), e187-94.
[86] Sandhya Vasudevan, William C. Vogt, Sandy Weininger, and T. Joshua Pfefer. 2024. Melanometry for objective evaluation of skin pigmentation in pulse oximetry studies. Communications Medicine 4, 1 (2024), 138. doi:10.1038/s43856-024-00550-7
[87] F. von Luschan. 1897. Beiträge zur Völkerkunde der Deutschen Schutzgebieten. Deutsche Buchgemeinschaft, Berlin.
[88] M. Wald, P. Erwin, N. Annon-Eberharter, and T. Werther. 2023. LED light can falsify pulse oximetry readings via the stroboscopic effect. Phys Eng Sci Med 46, 4 (December 2023), 1667-1675. doi:10.1007/s13246-023-01328-2 Epub 2023 Sep. 19.
[89] Amanda Watson, Claire Kendell, Anush Lingamoorthy, Insup Lee, and JamesWeimer. 2023. Lumos: An Open-Source Device for Wearable Spectroscopy Research. Proc. ACM Interact. Mob. Wearable Ubiquitous Technol. 6, 4, Article 187 (January 2023), 24 pages. doi:10.1145/3569502
[90] S. Westland. 2016. The CIE System. In Handbook of Visual Display Technology, J. Chen, W. Cranton, and M. Fihn (Eds.). Springer, Cham. doi: 10.1007/978-3-319-14346-0_11
[91] Ross D Whitehead, Daniel Re, Dengke Xiao, Gozde Ozakinci, and David I Perrett. 2012. You are what you eat: within-subject increases in fruit and vegetable consumption confer beneficial skin-color changes. PLoS One 7, 3 (2012), e32988. doi:10.1371/journal.pone.0032988
[92] Antony R Young. 1997. Chromophores in human skin. Physics in Medicine & Biology 42, 5 (1997), 789. doi: 10.1088/0031-9155/42/5/004
[93] George Zonios, Julie Bykowski, and Nikiforos Kollias. 2001. Skin Melanin, Hemoglobin, and Light Scattering Properties can be Quantitatively Assessed In Vivo Using Diffuse Reflectance Spectroscopy. Journal of Investigative Dermatology 117, 6 (2001), 1452-1457.
[94] George Zonios, Aikaterini Dimou, Ioannis Bassukas, Dimitrios Galaris, Argyrios Tsolakidis, and Efthimios Kaxiras. 2008. Melanin absorption spectroscopy: new method for noninvasive skin investigation and melanoma detection. Journal of Biomedical Optics 13, 1 (January 2008), 014017. doi:10.1117/1.2844710

1. A system for collecting skin tone data for a subject, the system comprising:
a plurality of light emitting diodes (LEDs) directing a range of light wavelengths onto skin;
a corresponding set of photodiodes (PDs) capturing spectral data for a corresponding range of wavelengths from reflected light from the skin;
a computer comprising a processor connected to computer memory storing software that when executed performs a computer implemented method comprising steps of:
extracting features from the spectral data with a signal processing component of the software;
applying the features from the spectral data to a machine learning model that identifies physiology data and color tone data for the skin.

2. The system of claim 1, wherein the photodiodes capture the spectral data on a continuous basis.

3. The system of claim 1, wherein extracting features comprises calculating mean values of each PD response over a measurement period.

4. The system of claim 1, wherein extracting features comprises calculating direct current (DC) content of each PD response over a measurement period.

5. The system of claim 1, wherein extracting features comprises calculating an individual typology angle (ITA) for the skin.

6. The system of claim 1, wherein extracting features comprises calculating ratios of combinations of LED outputs and PD responses.

7. The system of claim 6, wherein the combinations comprise different wavelengths of light.

8. The system of claim 1, wherein the range of wavelengths of the LEDs is within the visible spectrum.

9. The system of claim 1, wherein the spectral data is three-dimensional spectral data.

10. The system of claim 1, wherein the three-dimensional spectral data comprises an L* axis, and a* axis, and a b* axis.

11. The system of claim 10, wherein extracting features comprises calculating an individual typology angle (ITA) for the skin with L* and b* components from the three-dimensional spectral data.

12. The system of claim 11, wherein the ITA is used to classify the color tone of the skin according to an ITA range.

13. The system of claim 10, wherein the machine learning model identifies the physiology data of the skin with a regression algorithm.

14. The system of claim 13, wherein the regression algorithm assigns a quantitative value to either or both of melanin level or erythema level of the skin.

15. The system of any one of claims 1, further comprising a pulse oximeter that is calibrated according to at least one of a skin color tone, a melanin level, or an erythema level.
